(19) United States
(12) Patent Application Publication
(10) Pub. No.: US 2025/0197838 A1
(43) Pub. Date: Jun. 19, 2025

(54) UROKINASE-TYPE PLASMINOGEN ACTIVATOR RECEPTOR BINDING PEPTIDES AND METHODS OF USE

(71) Applicants: The United States Government as represented by the Department of Veterans Affairs, Washington, DC (US); CASE WESTERN RESERVE UNIVERSITY, Cleveland, OH (US)

(72) Inventor: Evi X Stavrou, Shaker Heights, OH (US)

(21) Appl. No.: 18/849,711

(22) PCT Filed: Jan. 24, 2023
(86) PCT No.: PCT/US2023/061142

§ 371 Date: Sep. 23, 2024

Related U.S. Application Data
(60) Provisional application No. 63/323,306, filed on Mar. 24, 2022.

(51) Int. Cl. C12N 9/64; A61K 38/00; A61K 47/60; A61P 7/02; A61P 17/02; A61P 35/00

(52) U.S. Cl. CPC C12N 9/6451; A61K 47/60; A61P 7/02; A61P 17/02; A61P 35/00; A61K 38/00; C12Y 304/21038

(57) ABSTRACT

Disclosed herein, are peptides that bind urokinase-type plasminogen activator receptors. The peptides may comprise amino acid sequences of IPPWEAPK (SEQ ID NO: 1), DLAQCQTPTQAAPPTPVSPR (SEQ ID NO: 2), or LHVPLMPAQPAPPK (SEQ ID NO: 3), or retro-inverso amino acid sequences of the above enumerated sequences. Also described herein, are methods of administering compounds comprising peptides that bind urokinase-type plasminogen activator receptors to subjects for the treatment of ovarian cancer and improving wound closure.

### CROSS REFERENCE TO RELATED APPLICATIONS

This application claims the benefit of U.S. Provisional Application No. 63/323,306, filed Mar. 24, 2022. The content of this earlier filed application is hereby incorporated by reference herein in its entirety.

### STATEMENT REGARDING FEDERALLY FUNDED RESEARCH

This invention was made with government support under grant number 1101BX003851 awarded by the United States Department of Veterans Affairs. The government has certain rights in the invention.

### INCORPORATION OF THE SEQUENCE LISTING

The present application contains a sequence listing that is submitted via EFS-Web concurrent with the filing of this application, containing the file name “37759_0408P1_Sequence_Listing.xml” which is 20,480 bytes in size, created on Jan. 23, 2023, and is herein incorporated by reference in its entirety.

### SUMMARY

Disclosed herein are peptides comprising an amino acid sequence of at least 60% identity to the amino acid sequence of IPPWEAPK (SEQ ID NO: 1) or a retro-inverso amino acid sequence of SEQ ID NO: 1, wherein the peptide binds urokinase-type plasminogen activator receptor (uPAR).

Disclosed herein are peptides comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, wherein the peptide binds urokinase-type plasminogen activator receptor (uPAR).

Disclosed herein are peptides comprising an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR).

Disclosed herein are methods of reducing neutrophil activation in a subject, the method comprising: a) administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR); and b) a pharmaceutically acceptable carrier.

Disclosed herein are methods of reducing reaction oxygen species production in a subject, the method comprising: a) administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR); and b) a pharmaceutically acceptable carrier.

Disclosed herein are methods of reducing production of neutrophil extracellular traps in a subject, the method comprising: a) administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR); and b) a pharmaceutically acceptable carrier.

Disclosed herein are methods of improving wound closure in a subject, the method comprising: a) administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR); and b) a pharmaceutically acceptable carrier.

Disclosed herein are methods of reducing vimentin levels in a subject, the method comprising: a) administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR); and b) a pharmaceutically acceptable carrier.

Disclosed herein are methods of reducing epithelial-to-mesenchymal transition in a subject, the method comprising: a) administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR); and b) a pharmaceutically acceptable carrier. Other features and advantages of the present compositions and methods are illustrated in the description below; the drawings, and the claims.

### BRIEF DESCRIPTION OF THE DRAWINGS

FIGS. 1A-D show the characterization of the FXII-uPAR binding interphase. FIGS. 1A and 1B show that hydroxyl radical footprinting and mass spectrometry identified the uPAR binding sites on FXII which mapped in the Fibronectin Type II (FN II) region, kringle region (KR), and proline-rich region (PR). FIG. 1C shows the corresponding FXII binding sites on uPAR that were also identified (top panel). FIG. 1C also shows IPPWEAPK (SEQ ID NO: 1), DLAQCQTPTQAAPPTPVSPR (SEQ ID NO: 2), and LHVPLMPAQPAPPKPQPTTR (SEQ ID NO: 11). FIG. 1D shows microscale thermophoresis evaluated uPAR binding on site-directed FXII mutants.

FIGS. 2A-D show the characterization of FXII-derived inhibitory peptides. FIG. 2A shows microscale thermophoresis of FXII-uPAR binding, in the absence or presence of FXII-derived inhibitory peptides. FIGS. 2B-C shows FXII-derived peptides significantly interfere with FXII-uPAR-mediated pAkt2 formation (FIG. 2B) and reactive oxygen species (ROS) generation (FIG. 2C). FXII_fl: recombinant full length FXII: Combo: combination of IPP-DLA-LHV peptides, 10 μM each. * p=0.004 FXII_fl vs. Combo, 2-way ANOVA. FIG. 2D shows ex vivo wound confluence of skin epithelial cells (SECs), in the absence or presence of murine wild type neutrophils & combination FXII peptides.

FIG. 3 shows a schematic of thromboinflammation in deep vein thrombosis (DVT). In response to flow restriction, neutrophils (Neu) and platelets establish heterotypic cell-cell interactions. Activated cells release Zinc (Zn2+), an important step for FXII to engage its receptor uPAR on neutrophils and promote Akt2 phosphorylation (pAkt2). pAkt2 enhances αMβ2 surface expression and promotes neutrophil extracellular trap (NET) formation. These events support additional neutrophil-platelet interactions, fibrin formation and propagation of DVT.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified. or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and new born subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for a disease caused by neutrophil activation or inflammatory diseases accompanied by neutrophil activation, such as, for example, prior to the administering step.

“Treatment” and “treating” refer to administration or application of a therapeutic agent (e.g., a peptide or polypeptide described herein) to a subject or performance of a procedure or modality on a subject for the purpose of obtaining a therapeutic benefit of a disease or health-related condition. For example, a treatment may include administration of a pharmaceutically effective amount of a peptide or polypeptide that binds urokinase-type plasminogen activator receptor (uPAR).

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be a disease caused by neutrophil activation or inflammatory diseases accompanied by neutrophil activation.

The terms “preventing,” “blocking,” “antagonizing,” or “reversing” mean preventing in whole or in part, or ameliorating or controlling.

The terms “diminishing,” “reducing,” or “preventing,” “inhibiting,” and variations of these terms, as used herein include any measurable decrease, including complete or substantially complete inhibition. The terms “enhance” or “enhanced” as used herein include any measurable increase or intensification.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step. As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the(S) configuration. In some aspects, the amino acids are in the D- or L-configuration. If non-naturally occurring side chains are used, non-amino acid substituents may be used, for example to prevent or retard in vivo degradation.

As used herein, the term “polypeptide” refers to a polymer composed of amino acid residues related naturally occurring structural variants, and synthetic non-naturally occurring analogs thereof linked via peptide bonds or modified peptide bonds (i.e., peptide isosteres), related naturally occurring structural variants, and synthetic non-naturally occurring analogs thereof, glycosylated polypeptides, and all “mimetic” and “peptidomimetic” polypeptide forms. Synthetic polypeptides can be synthesized, for example, using an automated polypeptide synthesizer. The term can refer to an oligopeptide, peptide, polypeptide, or protein sequence, or to a fragment, portion, or subunit of any of these. The term “protein” typically refers to large polypeptides. The term “peptide” typically refers to short polypeptides.

A “portion” of a polypeptide or protein means at least about three sequential amino acid residues of the polypeptide. It is understood that a portion of a polypeptide may include every amino acid residue of the polypeptide.

The term “fragment” can refer to a portion (e.g., at least 5, 10, 25, 50, 100, 125, 150, 200, 250, 300, 350, 400 or 500, etc. amino acids or nucleic acids) of a peptide that is substantially identical to a reference peptide and retains the biological activity of the reference peptide. In some aspects, the fragment or portion of a peptide retains at least 50%, 75%, 80%, 85%, 90%, 95% or 99% of the biological activity of the reference peptide described herein. A fragment of a referenced peptide can be a continuous or contiguous portion of the referenced polypeptide (e.g., a fragment of a reference peptide that is ten amino acids long can be any 2-9 contiguous residues within that reference peptide).

“Mutants,” “derivatives,” and “variants” of a polypeptide (or of the nucleic acid encoding the same) are polypeptides (or the nucleic acids) which may be modified or altered in one or more amino acids (or in one or more nucleotides) such that the peptide (or the nucleic acid) is not identical to the wild-type sequence, but has homology to the wild type polypeptide (or the nucleic acid).

The term “variant” can refer to a peptide or gene product that displays modifications in sequence and/or functional properties (i.e., altered characteristics) when compared to the wild-type peptide or gene product. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level. In an aspect, the term “variant” can mean a difference in some way from the reference sequence other than just a simple deletion of an N- and/or C-terminal amino acid residue or residues. In an aspect, a variant can include a substitution of an amino acid residue, the substitution can be considered conservative or non-conservative. Conservative substitutions are those within the following groups: Ser, Thr, and Cys: Leu, lie, and Val: Glu and Asp: Lys and Arg: Phe, Tyr, and Trp; and Gin, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂), COOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe: citrulline and methionine sulfoxide are neutral nonpolar, cysteic acid is acidic, and ornithine is basic. Proline may be substituted with hydroxyproline and retain the conformation conferring properties of proline.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more, and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As used herein, the terms “PEG”, “polyethylene glycol”, or “poly(ethylene glycol)” as used herein refers to any water soluble poly(ethylene oxide), and includes molecules comprising the structure —(CH₂CH₂O)ₙ— where n is an integer from 2 to about 800. A commonly used PEG is end-capped PEG, wherein one end of the PEG is capped with a relatively inactive group such as an alkoxy while the other end is a hydroxyl group that may be further modified. An often-used capping group is methoxy and the corresponding end-capped PEG is often denoted mPEG. The notion PEG is often used instead of mPEG. Specific PEG forms of the invention are branched, linear, forked PEGs, and the like and the PEG groups are typically polydisperse, possessing a low polydispersity index of less than about 1.05. The PEG moieties of the invention will, for a given molecular weight, typically consist of a range of ethylene glycol (or ethyleneoxide) monomers. For example, a PEG moiety of molecular weight 2000 Da will typically consist of 43+10 monomers, the average being around 43 monomers. The term “PEGylated” refers to the covalent attachment of PEG to another molecule, such as any of the peptides disclosed herein.

As used herein, the term “fatty acid” includes saturated fatty acids, which do not contain any double or triple bonds in the hydrocarbon chain. Saturated fatty acids include, but are not limited to propionic acid (C3) (by way of example, C3 indicates propionic acid has 3 carbon atoms in its hydrocarbon chain: the number of carbon atoms in the hydrocarbon chain of other example fatty acids is denoted in analogous fashion herein), butyric acid (C4), valeric acid (C5), caproic acid (C6), enanthic acid (C7), caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), isostearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), heptacosylic acid (C27), montanic acid (C28), nonacocylic acid (C29), melissic acid (C30), henatriacontylic acid (C31), lacceroic acid (C32), psyllic acid (C33), geddic acid (C34), ceroplastic acid (C35) and hexatriacontylic acid (C36).

As used herein, the term “fatty acid” also includes monounsaturated fatty acids, which contain one double or triple bond in the hydrocarbon chain, and polyunsaturated fatty acids, which contain more than one double and/or triple bond in the hydrocarbon chain. Such acids include, but are not limited to the omega 3, omega 6, omega 9 fatty acids, other fatty acids such as myristoleic and palmitoleic acid and conjugated fatty acids. Examples of monounsaturated and polyunsaturated fatty acids include but are not limited to, (a) omega 3 fatty acids, such as hexadecatrienoic acid (C16:3): (by way of example, C16:3 indicates hexadecatrienoic acid has 16 carbon atoms in its hydrocarbon chain and 3 double bonds; the number of carbon atoms and double bonds in the hydrocarbon chain of other example unsaturated fatty acids is denoted in analogous fashion herein), alpha linolenic acid (C18:3) and eicosapentanoic acid (20:5), (b) omega 6 fatty acids, such as linoleic acid (18:2), docosadienoic acid (C22:2), arachidonic acid (C20:4) and tetracosatetraenoic acid (C24:5), (c) omega 9 fatty acids, such as oleic acid (C18:1), eicosenoic acid (C20:1) and nevronic acid (C24:1), and (d) conjugated fatty acids such as rumenic acid (C18:2), eleostatic acid (C18:3), and rumelenic acid (C18:3).

The term “ω-amino-fatty acid” refers to fatty acids which feature an amino group at the distal carbon of the hydrocarbon chain thereof. The ω-amino-fatty acid moieties that are used in the context of the present invention can be saturated or unsaturated hydrocarbon chains. These moieties have a carboxylic group at one end of the hydrocarbon chain and an amine group at the other. The hydrocarbon chain connecting the carboxylic and amine groups in such an ω-amino-fatty acid moiety typically has from 3 to 32 carbon atoms.

Exemplary ω-amino-fatty acids include, without limitation, 4-amino-butyric acid, 6-amino-caproic acid, 8-amino-caprylic acid, 10-amino-capric acid (10-amino-decanoic acid), 12-amino-lauric acid (12-amino-dodecanoic acid), 14-amino-myristic acid (14-amino-tetradecanoic acid), 14-amino-myristoleic acid, 16-amino-palmitic acid (16-amino-hexadecanoic acid), 18-amino-stearic acid, 18-amino-oleic acid, 16-amino-palmitoleic acid, 18-amino-linoleic acid, 18-amino-linolenic acid and 20-amino-arachidonic acid.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### Compositions

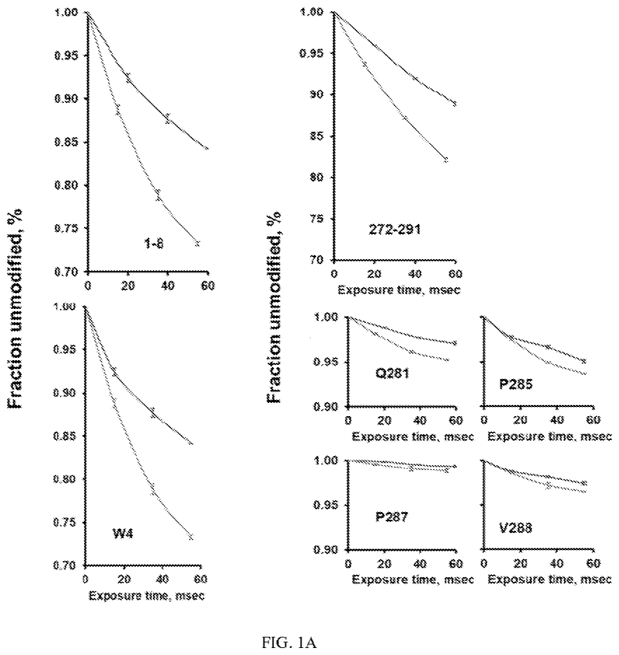

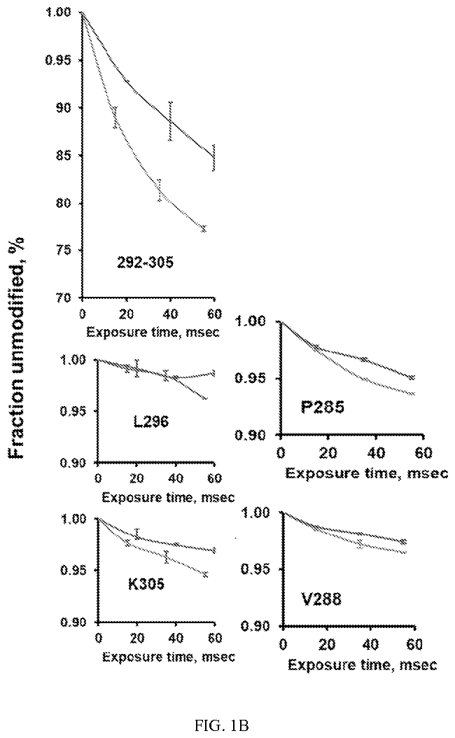

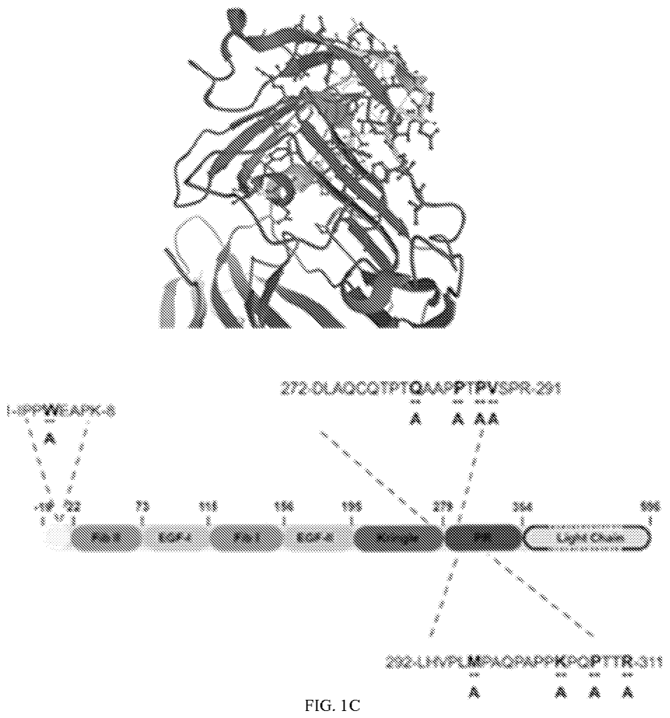

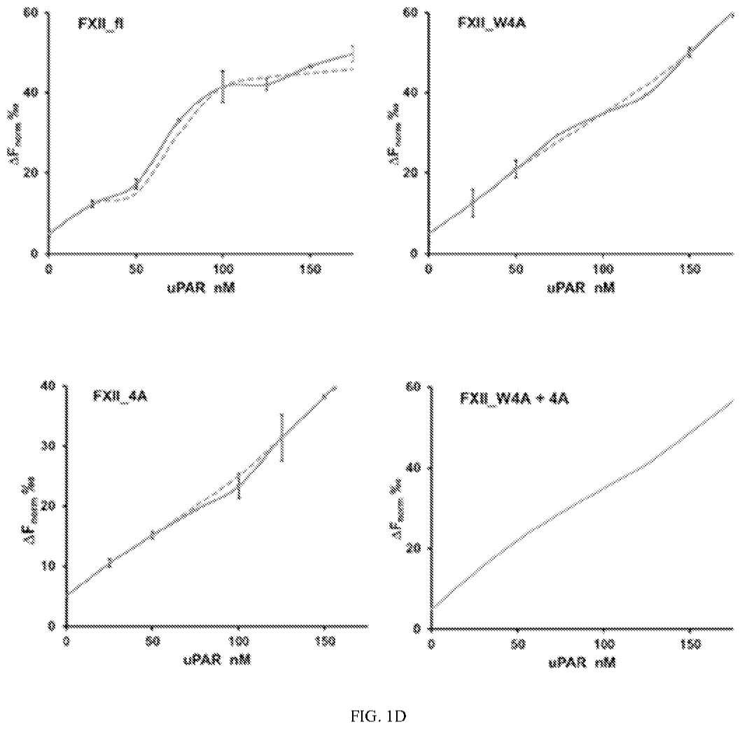

Disclosed herein are peptides and compositions comprising or consisting of peptides that bind to the urokinase-type plasminogen activator receptor (uPAR). In some aspects, the peptides and compositions disclosed herein can bind to the Fibronectin Type II (FN II) region, kringle region (KR) or the proline-rich region (PR) of the urokinase-type plasminogen activator receptor (uPAR). FIG. 1 shows exemplary uPAR binding sites that the peptides and compositions disclosed herein can bind.

Also disclosed herein are peptides comprising or consisting of an amino acid sequence of at least 60% identity to the amino acid sequence of IPPWEAPK (SEQ ID NO: 1) or a retro-inverso amino acid sequence of SEQ ID NO: 1, wherein the peptide binds urokinase-type plasminogen activator receptor (uPAR). Further disclosed herein are peptides comprising or consisting of an amino acid sequence of at least 70% identity to the amino acid sequence of IPPWEAPK (SEQ ID NO: 1) or a retro-inverso amino acid sequence of SEQ ID NO: 1. In some aspects, the peptides disclosed herein comprise or consist of an amino acid sequence of at least 80% identity to the amino acid sequence of IPPWEAPK (SEQ ID NO: 1) or a retro-inverso amino acid sequence of SEQ ID NO: 1. In some aspects, the peptides disclosed herein comprise or consist of an amino acid sequence of at least 90% identity to the amino acid sequence of IPPWEAPK (SEQ ID NO: 1) or a retro-inverso amino acid sequence of SEQ ID NO: 1. In some aspects, the peptides disclosed herein comprise or consist of an amino acid sequence of at least 91%, 92%, 93%, 94%, 95%, 96%, 97%, 98%, 99% or 100% identity to the amino acid sequence of IPPWEAPK (SEQ ID NO: 1) or a retro-inverso amino acid sequence of SEQ ID NO: 1. In some aspects, the peptide comprises an amino acid sequence comprising a W at position 4 of SEQ ID NO: 1.

Disclosed herein are peptides comprising or consisting of an amino acid sequence of X₁X₂X₃W X₅X₆X₇X₈ (SEQ ID NO: 4) or a retro-inverso amino acid sequence of SEQ ID NO: 4, wherein the peptide binds urokinase-type plasminogen activator receptor (uPAR), and wherein the amino acid sequence is at least 60% identical to the amino acid sequence of IPPWEAPK (SEQ ID NO: 1). In some aspects, the peptides disclosed herein can comprise a substitution of at least one amino acid of at least one to three of residues 11, 2P, 3P, 5E, 6A, 7P, or 8K of IPPWEAPK (SEQ ID NO: 1). In some aspects, the peptide will not comprise any substitution at amino acid 4W of IPPWEAPK (SEQ ID NO: 1).

Also disclosed herein are peptides comprising or consisting of an amino acid sequence of at least 60% identity to the amino acid sequence of DLAQCQTPTQAAPPTPVSPR (SEQ ID NO: 2) or a retro-inverso amino acid sequence of SEQ ID NO: 2, wherein the peptide binds urokinase-type plasminogen activator receptor (uPAR). Further disclosed herein are peptides comprising or consisting of an amino acid sequence of at least 70% identity to the amino acid sequence of DLAQCQTPTQAAPPTPVSPR (SEQ ID NO: 2) or a retro-inverso amino acid sequence of SEQ ID NO: 2. In some aspects, the peptides disclosed herein comprise or consist of an amino acid sequence of at least 80% identity to the amino acid sequence of DLAQCQTPTQAAPPTPVSPR (SEQ ID NO: 2) or a retro-inverso amino acid sequence of SEQ ID NO: 2. In some aspects, the peptides disclosed herein comprise or consist of an amino acid sequence of at least 90% identity to the amino acid sequence of DLAQCQTPTQAAPPTPVSPR (SEQ ID NO: 2) or a retro-inverso amino acid sequence of SEQ ID NO: 2. In some aspects, the peptides disclosed herein comprise or consist of an amino acid sequence of at least 91%, 92%, 93%, 94%, 95%, 96%, 97%, 98%, 99% or 100% identity to the amino acid sequence of DLAQCQTPTQAAPPTPVSPR (SEQ ID NO: 2) or a retro-inverso amino acid sequence of SEQ ID NO: 2. In some aspects, the peptide comprises an amino acid sequence comprising a Q at position 10, a P at position 14, a P at position 16, and a V at position 17 of SEQ ID NO: 2. In some aspects, the peptide will not comprise any substitution at the amino acid Q at position 10, P at position 14, P at position 16, or V at position 17 of SEQ ID NO: 2.

Disclosed herein are peptides comprising or consisting of an amino acid sequence of DLAQCQTPTX₁AAPX₂TX₃X₄SPR (SEQ ID NO: 5) or a retro-inverso amino acid sequence of SEQ ID NO: 5, wherein the peptide binds urokinase-type plasminogen activator receptor (uPAR), and wherein the amino acid sequence is at least 60% identical to the amino acid sequence of DLAQCQTPTQAAPPTPVSPR (SEQ ID NO: 2). In some aspects, the peptides disclosed herein can comprise a substitution of at least one amino acid of at least one to eight of residues ID, 2L, 3A, 4Q, 5C, 6Q, 7T, 8P, 9T, 11A, 12A, 13P, 15T, 18S, 19R, or 20R of DLAQCQTPTQAAPPTPVSPR (SEQ ID NO: 2). In some aspects, the peptide will not comprise any substitutions at amino acid 10Q, 14P, 16P, and 17V of DLAQCQTPTQAAPPTPVSPR (SEQ ID NO: 2).

Also disclosed herein are peptides comprising or consisting of an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR). Further disclosed herein are peptides comprising or consisting of an amino acid sequence of at least 70% identity to the amino acid sequence of LHVPLMPAQPAPPK (SEQ ID NO: 3) or a retro-inverso amino acid sequence of SEQ ID NO: 3. In some aspects, the peptides disclosed herein comprise or consist of an amino acid sequence of at least 80% identity to the amino acid sequence of LHVPLMPAQPAPPK (SEQ ID NO: 3) or a retro-inverso amino acid sequence of SEQ ID NO: 3. In some aspects, the peptides disclosed herein comprise or consist of an amino acid sequence of at least 90% identity to the amino acid sequence of LHVPLMPAQPAPPK (SEQ ID NO: 3) or a retro-inverso amino acid sequence of SEQ ID NO: 3. In some aspects, the peptides disclosed herein comprise or consist of an amino acid sequence of at least 91%, 92%, 93%, 94%, 95%, 96%, 97%, 98%, 99% or 100% identity to the amino acid sequence of LHVPLMPAQPAPPK (SEQ ID NO: 3) or a retro-inverso amino acid sequence of SEQ ID NO: 3. In some aspects, the peptide comprises an amino acid sequence comprising a H at position 2, a V at position 3, a M at position 6, or a K at position 14 of SEQ ID NO: 3. In some aspects, the peptide will not comprise any substitution at a H at position 2, a V at position 3, a M at position 6, or a K at position 14 of SEQ ID NO: 3.

Disclosed herein are peptides comprising or consisting of an amino acid sequence of LX₁X₂PX₃X₄PAQPAPPX₅ (SEQ ID NO: 6) or a retro-inverso amino acid sequence of SEQ ID NO: 5, wherein the peptide binds urokinase-type plasminogen activator receptor (uPAR), and wherein the amino acid sequence is at least 60% identical to the amino acid sequence of LHVPLMPAQPAPPK (SEQ ID NO: 3). In some aspects, the peptides disclosed herein can comprise a substitution of at least one amino acid. In some aspects, the peptides disclosed herein can comprise a substitution of at least one to eight of residues IL, 4P, 7P, 8A, 9Q, 10P, 11A, 12P, or 13P of LHVPLMPAQPAPPK (SEQ ID NO: 3). In some aspects, the peptide will not comprise any substitutions at amino acid 2H, 3V, 6M, and 14K of LHVPLMPAQPAPPK (SEQ ID NO: 3).

In some aspects, the substitution to any of the amino acids in any of the peptides disclosed herein can be charge-dependent. In some aspects, a large side chain can be included to maintain a structure that is important for forming a loop to the kringle region. In some aspects, the large side chain can be a substitution for an amino acid and/or attached to one of the amino acids of any of the peptides disclosed herein.

In some aspects, the peptides can comprise an amino acid sequence of at least 60% identity to the amino acid sequence of IPPWEAPK (SEQ ID NO: 1), at least 60% identity to the amino acid sequence of DLAQCQTPTQAAPPTPVSPR (SEQ ID NO: 2), at least 60% identity to the amino acid sequence of LHVPLMPAQPAPPK (SEQ ID NO: 3) or a combination thereof.

In some aspects, the peptides can comprise an amino acid sequence of at least 70% identity to the amino acid sequence of IPPWEAPK (SEQ ID NO: 1) or a retro-inverso amino acid sequence of SEQ ID NO: 1. In some aspects, the peptide can comprise a substitution of least one an amino acid of at least one of residue 11, 2P, 3P, 5E, 6A, 7P, or 8K of IPPWEAPK (SEQ ID NO: 1) or a retro-inverso amino acid sequence of SEQ ID NO: 1. In some aspects, the peptide can comprise an amino acid sequence of at least 60% identity to the amino acid sequence of DLAQCQTPTQAAPPTPVSPR (SEQ ID NO: 2) or a retro-inverso amino acid sequence of SEQ ID NO: 2. In some aspects, the peptide can comprise an amino acid sequence of at least 70% identity to the amino acid sequence of DLAQCQTPTQAAPPTPVSPR (SEQ ID NO: 2) or a retro-inverso amino acid sequence of SEQ ID NO: 2. In some aspects, the peptide can comprise a substitution of least one an amino acid of at least one of residue ID, 2L, 3A, 4Q, 5C, 6Q, 7T, 8P, 9T, 11A, 12A, 13P, 15T, 18S. 19R, or 20R of DLAQCQTPTQAAPPTPVSPR (SEQ ID NO: 2) or a retro-inverso amino acid sequence of SEQ ID NO: 2. In some aspects, the peptide can comprise an amino acid sequence of at least 60% identity to the amino acid sequence of LHVPLMPAQPAPPK (SEQ ID NO: 3) or a retro-inverso amino acid sequence of SEQ ID NO: 3. In some aspects, the peptide can comprise an amino acid sequence of at least 70% identity to the amino acid sequence of LHVPLMPAQPAPPK (SEQ ID NO: 3) or a retro-inverso amino acid sequence of SEQ ID NO: 3. In some aspects, the peptide can comprise a substitution of least one an amino acid of at least one of residue IL, 4P, 7P, 8A, 9Q, 10P, 11A, 12P, or 13P of LHVPLMPAQPAPPK (SEQ ID NO: 3) or a retro-inverso amino acid sequence of SEQ ID NO: 3.

Disclosed herein are compositions comprising one or more of the peptides or fragments thereof described herein. In some aspects, the compositions can further comprise a pharmaceutically acceptable carrier. In some aspects, the pharmaceutically acceptable carrier can be lipid-based or a polymer-based colloid. Examples of colloids include liposomes, hydrogels, microparticles, nanoparticles and micelles. In some aspects, any of the peptides or fragments thereof can be encapsulated by a nanoparticle.

In some aspects, the disease caused by neutrophil activation or inflammatory diseases accompanied by neutrophil activation can be one or more of venous and arterial thrombosis, deep vein thrombosis and vascular thrombo-embolism (DVT+VTE), lupus, psoriasis, atherosclerosis, endometriosis, trauma, sickle cell disease and associated acute hemolytic crisis, acute chest syndrome and pulmonary thrombosis, pulmonary artery thrombosis, immunothrombosis, COVID-19 infection, thrombo-inflammation, chronic and diabetic wounds, post-operative wounds, trauma and related wounds, sepsis, acute respiratory distress syndrome, acute pancreatitis, acute pulmonary disorder, pulmonary disorder caused by the hemorrhagic shock, multiple organ failure, burn, multiple injury, idiopathic interstitial pulmonary fibrosis, cancer, cerebral trauma, spinal cord injury, neuropathic pain, cerebral infarction, cerebral vasospasm after the subarachnoid hemorrhage, epilepsy, status epilepticus, viral encephalitis, influenza-associated encephalopathy, inflammatory bowel disease, Kawasaki disease, multiple sclerosis, diabetic vascular complication, diabetic wounds, hepatitis, arteriosclerosis, asthma bronchial, chronic bronchitis, pulmonary emphysema, organ dysfunction after surgical operation, organ dysfunction after radiotherapy, nephritis, nephrotic syndrome, acute renal failure, hemodialysis, extracorporeal circulation, artificial breathing, acute/chronic rejection after organ transplantation, systemic lupus erythematosus (SLE), rheumatoid arthritis, disseminated intravascular coagulation (DIC), autoimmune disease group, Bechet's disease, myocarditis, endocarditis, ischemia reperfusion disorder, myocardial infarction, congestive heart failure, adipose tissue inflammation, neutrophilic dermatosis, Sweet's disease, Stevens-Johnson syndrome, Reye syndrome, cachexia, chronic fatigue syndrome and fibromyalgia. In some aspects, the cancer can be the cancer is ovarian cancer, breast cancer, pancreatic cancer, prostate cancer, lung cancer, hepatocellular carcinoma, acute myeloid leukemia, acute lymphoblastic leukemia, non-Hodgkin lymphomas, or Hodgkin lymphoma.

The peptides and fragments thereof disclosed herein can be subject to various changes, substitutions, insertions, and deletions where such changes provide for certain advantages in its use. In some aspects, the peptides and fragments thereof disclosed herein that bind uPAR to can be substantially homologous with, rather than be identical to, the sequence of a recited peptide where one or more changes are made and it retains the ability to function as specifically binding to and/or complexing with urokinase-type plasminogen activator receptor (uPAR).

The peptides and fragments thereof disclosed herein can be in any of a variety of forms of polypeptide derivatives, including but not limited to amides, conjugates with proteins, cyclized polypeptides, polymerized polypeptides, retro-inverso peptides, analogs, fragments, chemically modified polypeptides, and the like derivatives.

In some aspects, the peptides disclosed herein can be linear.

In some aspects, the peptides and fragments thereof disclosed herein can be cyclized. In some aspects, the peptides and fragments thereof disclosed herein can be cyclized via a disulfide bridge between terminal cysteine residues. The peptides disclosed herein can include at least two cysteine residues, one or both of which are, optionally, at the C-terminal or N-terminal of the peptide. In some aspects, the peptides and fragments thereof can be cyclized by formation of a disulfide bond between these two cysteine residues (or, more generally, between two of the at least two cysteine residues present at the terminal regions). While the peptides and fragments thereof may be linear or cyclic, cyclic peptides generally have an advantage over linear peptides in that their cyclic structure is more rigid and hence their biological activity may be higher than that of the corresponding linear peptide. Any method for cyclizing peptides can be applied to the peptides and fragments thereof described herein. In some aspects, sortase-mediated cyclization or butelase-mediated cyclization methodologies.

Retro-inverso peptides are linear peptides whose amino acid sequence is reversed and the α-center chirality of the amino acid subunits is inverted as well. These types of peptides are designed by including D-amino acids in the reverse sequence to help maintain side chain topology similar to that of the original L-amino acid peptide and make them more resistant to proteolytic degradation. D-amino acids represent conformational mirror images of natural L-amino acids occurring in natural proteins present in biological systems. Peptides that contain D-amino acids have advantages over peptides that just contain L-amino acids. In general, these types of peptides are less susceptible to proteolytic degradation and have a longer effective time when used as pharmaceuticals. Furthermore, the insertion of D-amino acids in selected sequence regions as sequence blocks containing only D-amino acids or in-between L-amino acids allows the design of peptide based drugs that are bioactive and possess increased bioavailability in addition to being resistant to proteolysis. Furthermore, if properly designed, retro-inverso peptides can have binding characteristics similar to L-peptides.

The term “analog” includes any polypeptide having an amino acid residue sequence substantially identical to a sequence specifically shown herein in which one or more residues have been conservatively substituted with a functionally similar residue and that specifically binds to and/or complexes with the urokinase-type plasminogen activator receptor (uPAR) as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

“Chemical derivative” refers to a subject peptide or polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acids. For example, 4-hydroxyproline may be substituted for proline: 5-hydroxylysine may be substituted for lysine: 3-methylhistidine may be substituted for histidine: homoserine may be substituted for serine; and ornithine may be substituted for lysine. Peptides described herein also include any peptide having one or more additions and/or deletions or residues relative to the sequence of a peptide whose sequence is shown herein, so long as the requisite activity is maintained.

The term “fragment” or “fragment thereof” refers to any subject peptide or polypeptide having an amino acid residue sequence shorter than that of a peptide or polypeptide whose amino acid residue sequence is described herein.

Any peptide, polypeptide or compound can also be used in the form of a pharmaceutically acceptable salt. Acids, which are capable of forming salts with the peptides and polypeptides, include but are not limited to inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the peptides and polypeptides include inorganic bases such as sodium hydroxide, ammonium hydroxide, potassium hydroxide and the like; and organic bases such as mono-, di- and tri-alkyl and aryl-amines (e.g., triethylamine, diisopropylamine, methylamine, dimethylamine, and the like) and optionally substituted ethanolamines (e.g., ethanolamine, diethanolamine and the like).

The peptides and fragments thereof disclosed herein can be synthesized by any of the techniques that are known to those skilled in the art, including but not limited to recombinant DNA techniques. Synthetic chemistry techniques, such as a solid-phase Merrifield-type synthesis, can be used for reasons of purity, antigenic specificity, freedom from undesired side products, ease of production and the like. A summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976: J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie. “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different. selectively removable protecting group is utilized for amino acids containing a reactive side group such as lysine.

Using a solid phase synthesis as an example, the protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

In some aspects, the peptides and fragments thereof disclosed herein can be of any length so long as the binding of the peptides and fragments thereof disclosed herein to the uPAR receptor remains uninhibited. In some aspects, the peptides and fragments thereof disclosed herein can further comprise 1, 2, 3, 4, 5, 10, 15, 20, 25, 30, 35, 40, 45, 50 amino acid residues at the N-terminal end of the disclosed peptides. In some aspects, the peptides described herein can further comprise 1, 2, 3, 4, 5, 10, 15, 20, 25, 30, 35, 40, 45, 50 amino acid residues at the C-terminal end of the disclosed peptides disclosed herein. In some aspects, the amino acid residues that can be present at either the N-terminal end or the C-terminal end of any of the peptides disclosed herein can be unimportant for the binding of the peptides to uPAR 5. In some aspects, the amino acid residues added to the N-terminal end or the C-terminal end of the peptides disclosed herein may prevent ubiquitination, improve stability, help maintain the three dimensional structure of the peptide, or a combination thereof.

In some aspects, the peptides and fragments thereof disclosed herein disclosed herein can further comprise a peptide or polypeptide having one or more amino acid residues with a modified side chain. In some aspects, one or more amino acids of any of the peptides or polypeptides disclosed here can have a modified side chain. Examples of side chain modifications include but are not limited to modifications of amino acid groups, such as reductive alkylation: amidination with methylacetimidate: acylation with acetic anhydride: carbamolyation of amino groups with cynate: trinitrobenzylation of amino acid with 2,4,6-trinitrobenzene sulfonic acid (TNBS): alkylation of amino groups with succinic anhydride; and pyridoxylation with pridoxal-5-phosphate followed by reduction with NaBH₄.

In some aspects, the guanidine group of the arginine residue may be modified by the formation of a heterocyclic condensate using a reagent, such as 2,3-butanedione, phenylglyoxal, and glyoxal. In some aspects, the carboxyl group may be modified by carbodiimide activation via O-acylisourea formation, followed by subsequent derivatization, for example, to a corresponding amide.

In some aspects, the sulfhydryl group may be modified by methods, such as carboxymethylation with iodoacetic acid or iodoacetamide: performic acid oxidation with cysteic acid: formation of mixed disulfides by other thiol compounds: a reaction by maleimide, maleic anhydride, or other substituted maleimide: formation of mercury derivatives using 4-chloromercuribenzoate, 4-chloromercuriphenylsulfonic acid, phenylmercury chloride, 2-chloromercuri-4-nitrophenol, and other mercurial agents; and carbamolyation with cyanate at alkaline pH. In addition, the sulfhydryl group of cysteine may be substituted with a selenium equivalent, whereby a diselenium bond may be formed instead of at least one disulfide bonding site in the peptide.

In some aspects, the tryptophan residue may be modified by, for example, oxidation with N-bromosuccinimide or alkylation of the indole ring by 2-hydroxy-5-nitrobenzyl bromide or sulfonyl halide. Meanwhile, the tyrosine residue may be modified by nitration using tetranitromethane to form a 3-nitrotyrosine derivative.

In some aspects, the modification of the imidazole ring of the histidine residue may be accomplished by alkylation with an iodoacetic acid derivative or N-carbethoxylation with diethylpyrocarbonate.

In some aspects, the proline residue may be modified by, for example, hydroxylation at the 4-position.

In some aspects, the peptides and fragments thereof disclosed herein can be further modified to improve stability. In some aspects, any of the amino acid residues of the peptides described herein can be modified to improve stability. In some aspects, peptide can have at least one amino acid residue that has an acetyl group, a fluorenylmethoxy carbonyl group, a formyl group, a palmitoyl group, a myristyl group, a stearyl group, or polyethylene glycol. In some aspects, an acetyl protective group can be bound to the peptide described herein.

As used herein, the term “stability” refers to storage stability (e.g., room-temperature stability) as well as in vivo stability. The foregoing protective group can protect the peptides described herein from the attack of protein cleavage enzymes in vivo.

The peptides and fragments thereof disclosed herein can also include functional equivalents of the peptides described herein. As used herein, the term “functional equivalents” can refer to amino acid sequence variants having an amino acid substitution, addition, or deletion in some of the amino acid sequence of the peptide while simultaneously having similar or improved biological activity, compared with the peptide as described herein. In some aspects, the amino acid substitution can be a conservative substitution. Examples of the naturally occurring amino acid conservative substitution include, for example, aliphatic amino acids (Gly, Ala, and Pro), hydrophobic amino acids (Ile, Leu, and Val), aromatic amino acids (Phe, Tyr, and Trp), acidic amino acids (Asp and Glu), basic amino acids (His, Lys, Arg, Gln, and Asn), and sulfur-containing amino acids (Cys and Met). In some aspects, the amino acid deletion can be located in a region that is not directly involved in the activity of the peptide disclosed herein.

In some aspects, the amino acid sequence of the peptides and fragments thereof disclosed herein can include a peptide sequence that has substantial identity to any of the sequences of the peptides disclosed herein. As used herein, the term “substantial identity.” means that two amino acid sequences, when optimally aligned and then analyzed by an algorithm normally used in the art, such as BLAST, GAP, or BESTFIT, or by visual inspection, share at least about 60%, 70%, 80%, 85%, 90%, or 95% sequence identity. Methods of alignment for sequence comparison are known in the art.

In some aspects, the amino acid sequence of the peptides and fragments thereof disclosed herein can include a peptide sequence that has some degree of identity or homology to any of sequences of the peptides disclosed herein. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position: when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The peptides described herein can have at least or about 25%, 50%, 65%, 75%, 80%, 85%, 90%, 95%, 96%, 97%, 98%, or 99% identity or homology to the peptide or polypeptide, wherein the peptide is one or more of SEQ ID NOs: 1-6.

As discussed herein, there are numerous variants of the peptide that bind uPAR that are known and herein contemplated. Protein and peptide fragments, variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the peptide sequence. Typically, no more than about from 2 to 6 residues are deleted at any one site within the peptide. Amino acid substitutions are typically of single residues, but can occur at a number of different locations at once: insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative
Substitutions, others are known in the art.

ala; ser
arg; lys, gln
asn; gln; his
asp; glu
cys; ser
gln; asn, lys
glu; asp
gly; pro
his; asn; gln
ile; leu; val
leu; ile; val
lys; arg; gln;
met; leu; ile
phe; met; leu; tyr
ser; thr
thr; ser
trp; tyr
tyr; trp; phe
val; ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl: or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another or one polar residue for another. The substitutions include combinations such as, for example, Gly, Ala: Val, Ile, Leu: Asp, Glu: Asn, Gln: Ser, Thr: Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g., Arg, are accomplished for example by deleting one of the basic residues or substituting one by glutaminyl or histidyl residues.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

The degree of identity can vary and can be determined by methods well established in the art. “Homology” and “identity” each refer to sequence similarity between two polypeptide sequences, with identity being a stricter comparison. Homology and identity can each be determined by comparing a position in each sequence which may be aligned for purposes of comparison. When a position in the compared sequence is occupied by the same amino acid residue, then the polypeptides can be referred to as identical at that position: when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. A biologically active variant or a fragment of a peptide or polypeptide described herein can have at least or about 10%, 20%, 30%, 40%, 50%, 60%, 70%, 80%, 90%, 95%, 98%, or 99% identity or homology to a corresponding naturally occurring peptide or polypeptide.

In some aspects, the peptides described herein can include at the N- or C-termini, 1 to about 100 (e.g., 1, 2, 3, 4, 5, 6, 7, 8, 9, 10, 15, 20, 25, 30, 40, 50, 60, 70, 80, 90, or 100) amino acid residues that are positively charged (e.g., basic amino acid residues such as arginine, histidine, and/or lysine residues): 1 to about 100 amino acid residues that are negatively charged (e.g., acidic amino acid residues such as aspartic acid or glutamic acid residues): 1 to about 100 glycine residues: 1 to about 100 hydrophobic amino acid residues (e.g., hydrophobic aliphatic residues such as alanine, leucine, isoleucine or valine or hydrophobic aromatic residues such as phenylalanine, tryptophan or tyrosine): or 1 to about 100 (e.g., 1-4) cysteine residues. Where biologically active variants of an OCA-B fragment are used, the variant can vary by substitution of one or more amino acid residues within these groups. The variants can include a conservative amino acid substitution. In some aspects, the additional sequence(s) can be about 1 to 200 amino acid residues long, and these residues can be divided evenly or unevenly between the N- and C-termini. For example, both the N- and C-termini can include about 1, 10, 20, 30, 40, 50, 60, 70, 80, 90, or 100 amino acid residues. Alternatively, one terminus can include about 1, 10, 20, 30, 40, 50, 60, 70, 80, 90, 100, 110, 120, 130, 140, 150, 160, 170, 180, 190, or 200 residues, and one terminus can include none.

The peptides described herein and the fragments thereof, including the modified fragments described above as well as any variants disclosed herein, can be protease resistant and can include one or more types of protecting groups such as an acyl group, an amide group, a benzyl or benzoyl group, or a polyethylene glycol (PEG).

The peptides, fragments thereof and biologically active variants thereof can be modified in numerous ways. For example, agents, including additional amino acid residues, other substituents, and protecting groups can be added to either the amino terminus, the carboxy terminus, or both. The modification can be made for the purpose of altering the fragments' form or altering the way the fragments bind to or interact with other peptides or polypeptides. For example, the fragments can be modified to include cysteine residues or other sulphur-containing residues or agents that can participate in disulphide bond formation. For example, one can add at least one cysteine residue, one of which are, optionally, at the C-terminal or N-terminal of the fragment.

In some aspects, the peptides described herein can be linked or conjugated to a moiety at the N- or C-terminal ends. In some aspects, a reaction to link or conjugate the peptides disclosed here to a moiety can be: a) reaction of an amine with an NHS ester to form an amide bond: b) reaction of an amine with an aldehyde produces a Schiff base that can be reduced by borohydrides to produce a secondary amine linkage: c) reaction of a thiol (in a cysteine residue) with a maleimide derivative produces a thioether bond: d) reaction of a thiol-containing peptide with a vinylsulfone-modified PEG produces a thioether bond; and e) Cu-catalyzed alkyne-azide click reaction produces a triazole. In some aspects, the peptides described herein can be linked or conjugated to lipid nanoparticles. In some aspects, the method of linking or conjugated the peptides described herein to a

Linkers. The peptides described herein can also comprise one or more linkers. The linkers can be of any length, of a flexible sequence and not have any charges. In some aspects, the linker can be a peptide linker. In some aspects, the one or more linkers can be peptide-based. In some aspects, the one or more linkers can be GSG. In some aspects, the one or more linkers can be non-bulky amino acids. In some aspects, the one or more linkers can be AA, AAA, AGA, GGA, AGG, or GAG. In some aspects, the one or more linkers can be used combinatorially with serine.

In some aspects, the linker can be a covalent bond. To form covalent bonds, a chemically reactive group can be used, for instance, that has a wide variety of active carboxyl groups (e.g., esters) where the hydroxyl moiety is physiologically acceptable at the levels required to modify the peptide sequence or the peptide fragment sequence.

Any of the peptide sequences described herein and incorporated into the compounds can be modified to chemically interact with, or to include, a linker as described herein. These modified peptide sequences and peptide-linker constructs are within the scope of the present disclosure and can be packaged as a component of a kit with instructions for completing the process of conjugation. Conjugation refers to the coupling, linking, for example, through a covalent bond, connecting, associating two or more molecules. The peptide sequences can be modified to include a cysteine residue or other thio-bearing moiety (e.g., C-SH) at the N-terminus, C-terminus, or both.

Cyclized peptides. The peptides and fragments thereof disclosed herein can include at least two cysteine residues, one or both of which are, optionally, at the C-terminal or N-terminal of the peptide. For example, the peptide disclosed herein can have at or near the C- or N-termini, a cysteine residue. The peptide can be cyclized by formation of a disulfide bond between these two cysteine residues (or, more generally, between two of the at least two cysteine residues present at the terminal regions). While the peptides of the present disclosure may be linear or cyclic, cyclic peptides generally have an advantage over linear peptides in that their cyclic structure is more rigid and hence their biological activity may be higher than that of the corresponding linear peptide; and are stable such that lower doses or few administrations (e.g., injections) may be required. Any method for cyclizing peptides can be applied to the compounds described herein. In some aspects, sortase-mediated cyclization or butelase-mediated cyclization methodologies can be used.

Strategies for the preparation of circular polypeptides from linear precursors can be employed with the present peptides. For example, a chemical cross-linking approach can be used to prepare a backbone cyclized version of the peptide (Goldenburg and Creighton, J. Mol. Biol., 165:407-413, 1983). Other approaches include chemical intramolecular ligation methods (see, e.g., Camarero et al., Angew: Chern. Int. Ed., 37:347-349, 1998; Tam and Lu, Prot. Sci., 1:1583-1592, 1998; Camarero and Muir, Chern. Commun., 1997:1369-1370, 1997; and Zhang and Tam J. Am. Chern. Soc. 119:2363-2370, 1997) and enzymatic intramolecular ligation methods (Jackson et al., J. Am. Chern. Soc., 117:819-820, 1995), which allow linear synthetic peptides to be efficiently cyclized under aqueous conditions. See also U.S. Pat. No. 7,105,341.

PEGylation. In some aspects, the compounds disclosed herein can be PEGylated. In some aspects, the peptides disclosed herein can comprise one or more polyethylene glycol (PEG) moieties. PEGylation is a process of attaching the strands of the polymer PEG (polyethylene glycol) to molecules, including peptides. Said PEGylation can improve the safety and efficiency of the peptide. More specifically. PEGylation is the process of both covalent and non-covalent attachment or amalgamation of polyethylene glycol polymer chains to molecules and macrostructures, such as a drug, therapeutic protein or vesicles. PEGylation is routinely achieved by the incubation of a reactive derivative of PEG with the molecule. The covalent attachment of PEG to a drug or therapeutic protein can “mask” the agent from the host's immune system thereby reducing immunogenicity and antigenicity, and increasing the hydrodynamic size (size in solution) of the agent which prolongs its circulatory time by reducing renal clearance. PEGylation can also provide water solubility to hydrophobic drugs and proteins. In some aspects, the PEG molecules can have a variety of lengths and molecular weights, including, for example, PEG 200, PEG 1000, PEG 1500, PEG 4600, PEG 10,000, or combinations thereof. In some aspects, the PEG has a molecular weight of about 40 kDA to about 50 kDA.

In some aspects, one or more PEG moieties can be carboxylated PEG. In some aspects, the process of covalent attachment can be by click coupling of PEG (cycloaddition click reaction). In some aspects, one or more PEG moieties can be attached using on-resin coupling of PEG-CH₂—COOH to N-terminal peptide resins. In some aspects, the process of non-covalent binding of the peptides disclosed herein and PEG can be via reversible coupling, wherein the peptides can be tagged with a hexahistidine motif, which is recognized by the complementary nickel-nitriloacetic acid (Ni-NTA) complex on the end-modified PEG. In some aspects, PEG-peptide conjugates can be formed before they are cleaved from each other. In some aspects, PEG side-chain polymer-peptide conjugates can be synthesized using PEG-rich polymers bearing PEG or oligo-ethylene glycol side chains including but not limited to acrylates and methacrylates.

Fatty acids. In some aspects, the peptides and fragments thereof disclosed herein can comprise a fatty acid moiety. In some aspects, the fatty acid moiety is shown at the left side and is linked to the IPPWEAPK (SEQ ID NO: 1), DLAQCQTPTQAAPPTPVSPR (SEQ ID NO: 2), LHVPLMPAQPAPPK (SEQ ID NO: 3) or a retro-inverso amino acid of any of SEQ ID NOs: 1, 2, or 3. “EPA” indicates a moiety derived from 5,8, 11, 14, 17-eicosapentaenoic acid; and “DHA” indicates a moiety derived from 4,7,10,13,16, 19-docosahexaenoic acid.

In some aspects, the peptide can be any of the peptides and fragments thereof disclosed herein comprising an acetylated fatty acid.

Exemplary fatty acids from which a fatty acid moiety is derived include, without limitation, butyric acid, caproic acid, caprylic acid, capric acid, decanoic acid, lauric acid, myristic acid, palmitic acid, pentadecanoic acid, stearic acid, arachidic acid, behenic acid, erucic acid, lignoceric acid, margaric acid, myristoleic acid, palmitoleic acid, oleic acid, gadoleic acid, ricinoleic acid, vaccenic acid, linoleic acid, linolenic acid, alpha-linolenic acid, gamma-linolenic acid, licanic acid, margaroleic acid, arachidic acid, gadoleic acid, nervonic acid, arachidonic acid, docosapentaenoic (DPA), eicosapentaenoic acid (EPA), docosahexaenoic acid (DHA), and the like.

In some aspects, the peptide can be any of the peptides and fragments thereof disclosed herein comprising a saturated fatty acid. Exemplary saturated fatty acids include, but are not limited to, propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, and hexatriacontanoic acid.

In some aspects, the peptide can be any of the disclosed peptides comprising an unsaturated fatty acid. Exemplary unsaturated fatty acids include, but are not limited to, myristoleic acid, palmitoleic acid, sapienic acid, oleic acid, linoleic acid, α-linolenic acid, arachidonic acid, eicosapentaenoic acid (EPA), erucic acid, docosahexaenoic acid (DHA), and docosapentaenoic acid.

The peptides and polypeptides disclosed herein can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. (See Proteins-Structure and Molecular Properties 2nd Ed., T. E. Creighton, W. H. Freeman and Company, New York (1993): Posttranslational Covalent Modification of Proteins, B. C. Johnson, Ed., Academic Press, New York, pp. 1-12 (1983)).

In some aspects, any of IPPWEAPK (SEQ ID NO: 1), DLAQCQTPTQAAPPTPVSPR (SEQ ID NO: 2), LHVPLMPAQPAPPK (SEQ ID NO: 3) or a retro-inverso amino acid of any of SEQ ID NOs: 1, 2, or 3 can be conjugated to a fatty acid.

Labels. The peptides described herein can further comprise one or more labels or detection tags (e.g., FLAG™ tag, epitope or protein tags, such as myc tag, 6 His, and fluorescent fusion protein). In some aspects, the linker can be Fc or albumin. In some aspects, the label (e.g., FLAG™ tag) can be fused to the peptide. In some aspects, the disclosed methods and compositions can further comprise a fusion protein, or a polynucleotide encoding the same. In various aspects, the fusion protein comprises at least one epitope-providing amino acid sequence (e.g., “epitope-tag”), wherein the epitope-tag can be selected from i) an epitope-tag added to the N- and/or C-terminus of the peptide: or ii) an epitope-tag inserted into a region of the peptide, and an epitope-tag replacing a number of amino acids in the peptide.

Epitope tags are short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc. T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

In some aspects, the disclosed methods and compositions comprise an epitope-tag wherein the epitope-tag has a length of between 6 to 15 amino acids. In some aspects, the epitope-tag can have a length of 9 to 11 amino acids. The disclosed methods and compositions can also comprise a fusion protein comprising two or more epitope-tags, either spaced apart or directly in tandem. Further, the disclosed methods and composition can comprise 2, 3, 4, 5 or even more epitope-tags, as long as the fusion protein maintains its biological activity/activities (e.g., “functional”).

In some aspects, the epitope-tag can be a VSV-G tag, CD tag, calmodulin-binding peptide tag, S-tag, Avitag, SF-TAP-tag, strep-tag, myc-tag, FLAG-tag, T7-tag, HA (hemagglutinin)-tag. His-tag, S-tag, GST-tag, or GFP-tag. The sequences of these tags are described in the literature and well known to the person of skill in art.

As described herein, the term “immunologically binding” is a non-covalent form of attachment between an epitope of an antigen (e.g., the epitope-tag) and the antigen-specific part of an antibody or fragment thereof. Antibodies are preferably monoclonal and must be specific for the respective epitope tag(s) as used. Antibodies include murine, human and humanized antibodies. Antibody fragments are known to the person of skill and include, amongst others, single chain Fv antibody fragments (scFv fragments) and Fab-fragments. The antibodies can be produced by regular hybridoma and/or other recombinant techniques. Many antibodies are commercially available.

The construction of fusion proteins from domains of known proteins, or from whole proteins or proteins and peptides, is well known. Generally, a nucleic acid molecule that encodes the desired protein and/or peptide portions are joined using genetic engineering techniques to create a single, operably linked fusion oligonucleotide. Appropriate molecular biological techniques can be found in Sambrook et al. (Molecular Cloning: A laboratory manual Second Edition Cold Spring Harbor Laboratory Press, Cold spring harbor, NY, USA, 1989). Examples of genetically engineered multi-domain proteins, including those joined by various linkers, and those containing peptide tags, can be found in the following patent documents: U.S. Pat. No. 5,994,104 (“Interleukin-12 fusion protein”): U.S. Pat. No. 5,981,177 (“Protein fusion method and construction”); U.S. Pat. No. 5,914,254 (“Expression of fusion polypeptides transported out of the cytoplasm without leader sequences”): U.S. Pat. No. 5,856,456 (“Linker for linked fusion polypeptides”): U.S. Pat. No. 5,767,260 (“Antigen-binding fusion proteins”): U.S. Pat. No. 5,696,237 (“Recombinant antibody-toxin fusion protein”): U.S. Pat. No. 5,587,455 (“Cytotoxic agent against specific virus infection”): U.S. Pat. No. 4,851,341 (“Immunoaffinity purification system”): U.S. Pat. No. 4,703,004 (“Synthesis of protein with an identification peptide”); and WO 98/36087 (“Immunological tolerance to HIV epitopes”).

The placement of the functionalizing peptide portion (epitope-tag) within the subject fusion proteins or peptides can be influenced by the activity of the functionalizing peptide portion and the need to maintain at least substantial fusion protein, such as TCR, biological activity in the fusion. Two methods for placement of a functionalizing peptide are: N-terminal, and at a location within a protein portion that exhibits amenability to insertions. Though these are not the only locations in which functionalizing peptides can be inserted, they serve as good examples, and will be used as illustrations. Other appropriate insertion locations can be identified by inserting test peptide encoding sequences (e.g., a sequence encoding the FLAG peptide) into a construct at different locations, then assaying the resultant fusion for the appropriate biological activity and functionalizing peptide activity, using assays that are appropriate for the specific portions used to construct the fusion. The activity of the subject proteins can be measured using any of various known techniques, including those described herein.

In some aspects, any of the peptides or compositions disclosed herein can further include imaging agents. In some aspects, an imaging agents can include any substance that can be used for imaging or detecting a region of interest (ROI) in a subject and/or diagnosing the presence or absence of a disease or diseased tissue in a subject. The imaging agent can be used to generate a signal, which can be measured and whose intensity can related, and, in some aspects, be proportional, to the distribution of the imaging agent and activated platelets in the subject. Examples of imaging agents include, but are not limited to radionuclides, fluorescent dyes, chemiluminescent agents, colorimetric labels, and magnetic labels. In some aspects, the imaging agent can include a radiolabel that can be detected using gamma imaging wherein emitted gamma irradiation of the appropriate wavelength is detected. Methods of gamma imaging include, but are not limited to, SPECT and PET. For SPECT detection, the chosen radiolabel can lack a particular emission, but can produce a large number of photons in, for example, a 140-200 keV range. For PET detection, the radiolabel can be a positron-emitting moiety, such as 19F.

In some aspects, the imaging agent can include an MRS/MRI radiolabel, including but not limited to gadolinium, 19F, 13C, that can be coupled (e.g., attached or complexed) with the composition using general organic chemistry techniques. The imaging agent can also include radiolabels, such as 18F, 11C, 75Br, or 76Br for PET by techniques well known in the art and are described by Fowler, J. and Wolf, A. in Positron Emission Tomography and Autoradiography (Phelps, M., Mazziota, J., and Schelbert, H. eds.) 391-450) (Raven Press, NY 1986) the content of which is hereby incorporated by reference. The imaging can also include 1231 for SPECT.

In some aspects, the imaging agent can further include metal radiolabels. In some aspects, the radiolabel can be Technetium-99m (99mTc). Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26 (2): 217-24, (1999); Oya et al., “Small and neutral Tc (v)O BAT, bisaminoethanethiol (N2S2) complexes for developing new brain imaging agents”, Nuclear Medicine & Biology 25 (2): 135-40, (1998); and Hom et al., “Technetium-99m-labeled receptor-specific small-molecule radiopharmaceuticals: recent developments and encouraging results” Nuclear Medicine & Biology 24 (6): 485-98, (1997). Sequences. Sequences are shown in Table 3.

TABLE 3
Sequences.

| SEQ ID NO: | Sequence |
|---|---|
| 1 | IPPWEAPK |
| 2 | DLAQCQTPTQAAPPTPVSPR |
| 3 | LHVPLMPAQPAPPK |
| 7 | MRALLLLGFLLVSLESTLSIPPWEAPKEHK |
| | YKAEEHTVVLTVTGEPCHFPFQYHRQLYHK |
| | CTHKGRPGPQPWCATTPNFDQDQRWGYCLE |
| | PKKVKDHCSKHSPCQKGGTCVNMPSGPHCL |
| | CPQHLTGNHCQKEKCFEPQLLRFFHKNEIW |
| | YRTEQAAVARCQCKGPDAHCQRLASQACRT |
| | NPCLHGGRCLEVEGHRLCHCPVGYTGAFCD |
| | VDTKASCYDGRGLSYRGLARTTLSGAPCQP |
| | WASEATYRNVTAEQARNWGLGGHAFCRNPD |
| | NDIRPWCFVLNRDRLSWEYCDLAQCQTPTQ |
| | AAPPTPVSPRLHVPLMPAQPAPPKPQPTTR |
| | TPPQSQTPGALPAKREQPPSLTRNGPLSCG |
| | QRLRKSLSSMTRVVGGLVALRGAHPYIAAL |
| | YWGHSFCAGSLIAPCWVLTAAHCLQDRPAP |
| | EDLTVVLGQERRNHSCEPCQTLAVRSYRLH |
| | EAFSPVSYQHDLALLRLQEDADGSCALLSP |
| | YVQPVCLPSGAARPSETTLCQVAGWGHQFE |
| | GAEEYASFLQEAQVPFLSLERCSAPDVHGS |
| | SILPGMLCAGFLEGGTDACQGDSGGPLVCE |
| | DQAAERRLTLQGIISWGSGCGDRNKPGVYT |
| | DVAYYLAWIREHTVS |
| 8 | MGHPPLLPLLLLLHTCVPASWGLRCMQCKT |
| | NGDCRVEECALGQDLCRTTIVRLWEEGEEL |
| | ELVEKSCTHSEKTNRTLSYRTGLKITSLTE |
| | VVCGLDLCNQGNSGRAVTYSRSRYLECISC |
| | GSSDMSCERGRHQSLQCRSPEEQCLDVVTH |
| | WIQEGEEGRPKDDRHLRGCGYLPGCPGSNG |
| | FHNNDTFHFLKCC |
| 9 | NTTKCNEGPILELENLPQNGRQCYSCKGNS |
| | THGCSSEETFLIDCRGPMNQCLVATGTHEP |
| | KNQSYMVRGCATASMCQHAHLGDAFSMNHI |
| | DVSCCTKSGCNHPDLDVQYRSGAAPQPGPA |
| | HLSLTITLLMTARLWGGTLLWTIPPWEAPK |
| | EHKYKAEEHTVVLTVTGEPCHFPFQYHRQL |
| | YHKCTHKGRPGPQPWCATTPNFDQDQRWGY |
| | CLEPKKVKDHCSKHSPCQKGGTCVNMPSGP |
| | HCLCPQHLTGNHCQKEKCFEPQLLRFFHKN |
| | EIWYRTEQAAVARCQCKGPDAHCQRLASQA |
| | CRTNPCLHGGRCLEVEGHRLCHCPVGYTGA |
| | FCDVDTKASCYDGRGLSYRGLARTTLSGAP |
| | CQPWASEATYRNVTAEQARNWGLGGHAFCR |
| | NPDNDIRPWCFVLNRDRLSWEYCDLAQCQT |
| | PTQAAPPTPVSPRLHVPLMPAQPAPPKPQP |
| | TTRTPPQSQTPGALPAKREQPPSLTRNGPL |
| | SCGQRLRKSLSSMTRVVGGLVALRGAHPYI |
| | AALYWGHSFCAGSLIAPCWVLTAAHCLQDR |
| | PAPEDLTVVLGQERRNHSCEPCQTLAVRSY |
| | RLHEAFSPVSYQHDLALLRLQEDADGSCAL |
| | LSPYVQPVCLPSGAARPSETTLCQVAGWGH |
| | QFEGAEEYASFLQEAQVPFLSLERCSAPDV |
| | HGSSILPGMLCAGFLEGGTDACQGDSGGPL |
| | VCEDQAAERRLTLQGIISWGSGCGDRNKPG |
| | VYTDVAYYLAWIREHTVS |
| 10 | LRCMQCKTNGDCRVEECALGQDLCRTTIVR |
| | LWEEGEELELVEKSCTHSEKTNRTLSYRTG |
| | LKITSLTEVVCGLDLCNQGNSGRAVTYSRS |
| | RYLECISCGSSDMSCERGRHQSLQCRSPEE |
| | QCLDVVTHWIQEGEEGRPKDDRHLRGCGYL |
| | PGCPGSNGFHNNDTFHFLKCCNTTKCNEGP |
| | ILELENLPQNGRQCYSCKGNSTHGCSSEET |
| | FLIDCRGPMNQCLVATGTHEPKNQSYMVRG |
| | CATASMCQHAHLGDAFSMNHIDVSCCTKSG |
| | CNHPDLDVQYRSGAAPQPGPAHLSLTITLL |
| | MTARLWGGTLLWT |
| 11 | LHVPLMPAQPAPPKPQPTTR |

SEQ ID NO: 7 is coagulation Factor XII (/12. Homo sapiens). The signal peptide is underlined (amino acids 1-19) in Table 3: the numbering for the mature protein starts immediately thereafter (e.g., shown in bold: indicating the first amino acid of the mature protein without the signal peptide that is shed when FXII is secreted from cells). SEQ ID NO: 8 is uPAR (Plaur, Homo sapiens). The signal peptide is underlined (amino acids 1-22) in Table 3: the numbering for the mature protein starts immediately thereafter. SEQ ID NO: 9 is the amino acid sequence of the mature protein of coagulation Factor XII (F12, Homo sapiens). SEQ ID NO: 10 is the amino acid sequence of the mature protein of uPAR (Plaur, Homo sapiens).

### Pharmaceutical Compositions

As disclosed herein, are pharmaceutical compositions, comprising the peptides and fragments thereof and compositions comprising the peptides described herein and a pharmaceutical acceptable carrier. In some aspects, the pharmaceutical composition can be formulated for intravenous, subcutaneous, intradermal, intraperitoneal, intraocular, or intravitreal administration. The compositions of the present disclosure also contain a therapeutically effective amount of the peptides as described herein. The peptides and compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the peptides disclosed herein. Thus, compositions can be prepared for parenteral administration that includes the peptides dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical compositions described herein can also be formulated so as to provide slow, prolonged, or controlled release. For example, a controlled-release preparation is a pharmaceutical composition capable of releasing the peptides or compositions disclosed herein at a desired or required rate to maintain constant activity for a desired or required period of time.

### Methods of Treatment

Disclosed herein, are methods of treating a subject with a disease caused by neutrophil activation or inflammatory diseases accompanied by neutrophil activation. In some aspects, the methods can comprise: administering to the subject a therapeutically effective amount of any of the one or more of the peptides or fragments thereof disclosed herein. In some aspects, the treatment of the disease can require repression of FXII-uPAR-mediated pAkt2 formation or reactive oxygen species generation: repression, blockage or inhibition of FXII-mediated neutrophil activation: or blockage of the interaction between FXII with uPAR, the methods comprising: administering to a subject a therapeutically effective amount of any of the one or more of the peptides or fragments thereof disclosed herein. In some aspects, the methods of treating a subject with a disease caused by neutrophil activation or inflammatory diseases accompanied by neutrophil activation can comprise administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3. In some aspects, the methods can comprise a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3 and a pharmaceutically acceptable carrier. In some aspects, the peptide binds urokinase-type plasminogen activator receptor.

Disclosed herein, are methods of reducing neutrophil activation in a subject, the methods comprising: administering to the subject a therapeutically effective amount of any of the one or more of the peptides or fragments thereof disclosed herein. In some aspects, the methods of reducing neutrophil activation in a subject can comprise administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3. In some aspects, the methods can comprise a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3 and a pharmaceutically acceptable carrier. In some aspects, the peptide binds urokinase-type plasminogen activator receptor.

Disclosed herein, are methods of reducing reactive oxygen species production in a subject, the methods comprising: administering to the subject a therapeutically effective amount of any of the one or more of the peptides or fragments thereof disclosed herein. In some aspects, the methods of reducing reactive oxygen species production in a subject can comprise administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3. In some aspects, the methods can comprise a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3 and a pharmaceutically acceptable carrier. In some aspects, the peptide binds urokinase-type plasminogen activator receptor.

Disclosed herein, are methods of reducing producing of neutrophil extracellular traps in a subject, the methods comprising: administering to the subject a therapeutically effective amount of any of the one or more of the peptides or fragments thereof disclosed herein. In some aspects, the methods of reducing producing of neutrophil extracellular traps in a subject can comprise administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3. In some aspects, the methods can comprise a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3 and a pharmaceutically acceptable carrier. In some aspects, the peptide binds urokinase-type plasminogen activator receptor.

Disclosed herein, are methods of improving would closure in a subject, the methods comprising: administering to the subject a therapeutically effective amount of any of the one or more of the peptides or fragments thereof disclosed herein. In some aspects, the methods of improving would closure in a subject can comprise administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3. In some aspects, the methods can comprise a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3 and a pharmaceutically acceptable carrier. In some aspects, the peptide binds urokinase-type plasminogen activator receptor.

Disclosed herein, are methods of reducing vimentin levels in a subject, the methods comprising: administering to the subject a therapeutically effective amount of any of the one or more of the peptides or fragments thereof disclosed herein. In some aspects, the methods of reducing vimentin levels in a subject can comprise administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3. In some aspects, the methods can comprise a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3 and a pharmaceutically acceptable carrier. In some aspects, the peptide binds urokinase-type plasminogen activator receptor.

Disclosed herein, are methods of reducing epithelial-to-mesenchymal transition in a subject, the methods comprising: administering to the subject a therapeutically effective amount of any of the one or more of the peptides or fragments thereof disclosed herein. In some aspects, the methods of reducing epithelial-to-mesenchymal transition in a subject can comprise administering to the subject a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3. In some aspects, the methods can comprise a therapeutically effective amount of a peptide comprising an amino acid sequence of at least 60% identity to the amino acid sequence of IPPWEAPK (SEQ ID NO: 1) or a retro-inverso amino acid sequence of SEQ ID NO: 1, a peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, or a peptide comprising an amino acid sequence of at least 60% identity to the amino acid sequence of LHVPLMPAQPAPPK (SEQ ID NO: 3) or a retro-inverso amino acid sequence of SEQ ID NO: 3 and a pharmaceutically acceptable carrier. In some aspects, the peptide binds urokinase-type plasminogen activator receptor.

In some aspects, the methods disclosed herein can comprise identifying a patient in need of treatment before the administration step.

In some aspects, the methods disclosed herein comprise administering to the subject a therapeutically effective amount of any of the peptides disclosed herein and a pharmaceutically acceptable carrier or any of the compositions disclosed herein or any of the compositions comprising any of the peptides disclosed herein. In some aspects, the peptides bind urokinase-type plasminogen activator receptor (uPAR).

In some aspects, the peptides administered to the subject comprise: an amino acid sequence of at least 60% identity to the amino acid sequence of IPPWEAPK (SEQ ID NO: 1) or a retro-inverso amino acid sequence of SEQ ID NO: 1: an amino acid sequence of at least 70% identity to the amino acid sequence of IPPWEAPK (SEQ ID NO: 1) or a retro-inverso amino acid sequence of SEQ ID NO: 1: an amino acid sequence of at least 80% identity to the amino acid sequence of IPPWEAPK (SEQ ID NO: 1) or a retro-inverso amino acid sequence of SEQ ID NO: 1: an amino acid sequence of at least 90% identity to the amino acid sequence of IPPWEAPK (SEQ ID NO: 1) or a retro-inverso amino acid sequence of SEQ ID NO: 1: comprise an amino acid sequence of at least 91%, 92%, 93%, 94%, 95%, 96%, 97%, 98%, 99% or 100% identity to the amino acid sequence of IPPWEAPK (SEQ ID NO: 1) or a retro-inverso amino acid sequence of SEQ ID NO: 1: an amino acid sequence comprising a W at position 4 of SEQ ID NO: 1: a substitution of at least one amino acid of at least one to three of residues 11, 2P, 3P, 5E, 6A, 7P, or 8K of IPPWEAPK (SEQ ID NO: 1): will not comprise any substitutions at 4W of (SEQ ID NO: 1); an amino acid sequence of at least 60% identity to the amino acid sequence of DLAQCQTPTQAAPPTPVSPR (SEQ ID NO: 2) or a retro-inverso amino acid sequence of SEQ ID NO: 2: an amino acid sequence of at least 70% identity to the amino acid sequence of DLAQCQTPTQAAPPTPVSPR (SEQ ID NO: 2) or a retro-inverso amino acid sequence of SEQ ID NO: 2: an amino acid sequence of at least 80% identity to the amino acid sequence of DLAQCQTPTQAAPPTPVSPR (SEQ ID NO: 2) or a retro-inverso amino acid sequence of SEQ ID NO: 2: comprise an amino acid sequence of at least 90% identity to the amino acid sequence of DLAQCQTPTQAAPPTPVSPR (SEQ ID NO: 2) or a retro-inverso amino acid sequence of SEQ ID NO: 2: an amino acid sequence of at least 91%, 92%, 93%, 94%, 95%, 96%, 97%, 98%, 99% or 100% identity to the amino acid sequence of DLAQCQTPTQAAPPTPVSPR (SEQ ID NO: 2) or a retro-inverso amino acid sequence of SEQ ID NO: 2: an amino acid sequence comprising a Q at position 10, a P at position 14, a P at position 16, and a V at position 17 of SEQ ID NO: 2: a substitution of at least one amino acid of at least one to eight of residues ID, 2L, 3A, 4Q, 5C, 6Q, 7T, 8P, 9T, 11A, 12A, 13P, 15T, 18S, 19R, or 20R of DLAQCQTPTQAAPPTPVSPR (SEQ ID NO: 2): will not comprise any substitutions at the amino acid Q at position 10, P at position 14, P at position 16, or V at position 17 of SEQ ID NO: 2: an amino acid sequence of at least 60% identity to the amino acid sequence of LHVPLMPAQPAPPK (SEQ ID NO: 3) or a retro-inverso amino acid sequence of SEQ ID NO: 3: an amino acid sequence of at least 70% identity to the amino acid sequence of LHVPLMPAQPAPPK (SEQ ID NO: 3) or a retro-inverso amino acid sequence of SEQ ID NO: 3; an amino acid sequence of at least 80% identity to the amino acid sequence of LHVPLMPAQPAPPK (SEQ ID NO: 3) or a retro-inverso amino acid sequence of SEQ ID NO: 3; an amino acid sequence of at least 90% identity to the amino acid sequence of LHVPLMPAQPAPPK (SEQ ID NO: 3) or a retro-inverso amino acid sequence of SEQ ID NO: 3: an amino acid sequence of at least 91%, 92%, 93%, 94%, 95%, 96%, 97%, 98%, 99% 20) or 100% identity to the amino acid sequence of LHVPLMPAQPAPPK (SEQ ID NO: 3) or a retro-inverso amino acid sequence of SEQ ID NO: 3: an amino acid sequence comprising a H at position 2, a V at position 3, a M at position 6, or a K at position 14 of SEQ ID NO: 3: a substitution of at least one amino acid of at least one to eight of residues IL, 4P, 7P, 8A, 9Q, 10P. 11A, 12P, or 13P of LHVPLMPAQPAPPK (SEQ ID NO: 3): or will not comprise any substitutions at 2H, 3V, 6M, and 14K. In some aspects, the peptide will not comprise any substitution at a H at position 2, a V at position 3, a M at position 6, or a K at position 14 of SEQ ID NO: 3.

The pharmaceutical compositions described herein can be formulated to include a therapeutically effective amount of the peptides disclosed herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a disease caused by neutrophil activation or inflammatory diseases accompanied by neutrophil activation.

The pharmaceutical compositions described herein can be administered to the subject (e.g., a human patient) in an amount sufficient to delay, reduce, or preferably prevent the onset of clinical disease. Accordingly, in some aspects, the patient can be a human subject or patient. In therapeutic applications, compositions can be administered to a subject (e.g., a human patient) already with or diagnosed with a disease (e.g., a disease caused by neutrophil activation or inflammatory diseases accompanied by neutrophil activation) in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effect amount includes amounts that provide a treatment in which the onset or progression of the disease is delayed, hindered, or prevented, or the disease or a symptom of the disease is ameliorated. One or more of the symptoms can be less severe. Recovery can be accelerated in an individual who has been treated.

In some aspects, the disease caused by neutrophil activation or inflammatory diseases accompanied by neutrophil activation can be venous and arterial thrombosis, deep vein thrombosis and vascular thrombo-embolism (DVT+VTE), lupus, psoriasis, atherosclerosis, endometriosis, trauma, sickle cell disease and associated acute hemolytic crisis, acute chest syndrome and pulmonary thrombosis, immunothrombosis, COVID-19 infection, thrombo-inflammation, chronic and diabetic wounds, post-operative wounds, trauma and related wounds, sepsis, acute respiratory distress syndrome, acute pancreatitis, acute pulmonary disorder, pulmonary disorder caused by the hemorrhagic shock, multiple organ failure, burn, multiple injury, idiopathic interstitial pulmonary fibrosis, cancer, cerebral trauma, spinal cord injury, neuropathic pain, cerebral infarction, cerebral vasospasm after the subarachnoid hemorrhage, epilepsy, status epilepticus, viral encephalitis, influenza-associated encephalopathy, inflammatory bowel disease, Kawasaki disease, multiple sclerosis, diabetic vascular complication, diabetic wounds, hepatitis, arteriosclerosis, asthma bronchial, chronic bronchitis, pulmonary emphysema, organ dysfunction after surgical operation, organ dysfunction after radiotherapy, nephritis, nephrotic syndrome, acute renal failure, hemodialysis, extracorporeal circulation, artificial breathing, acute/chronic rejection after organ transplantation, systemic lupus erythematosus (SLE), rheumatoid arthritis, disseminated intravascular coagulation (DIC), autoimmune disease group, Bechet's disease, myocarditis, endocarditis, ischemia reperfusion disorder, myocardial infarction, congestive heart failure, adipose tissue inflammation, neutrophilic dermatosis, Sweet's disease, Stevens-Johnson syndrome, Reye syndrome, cachexia, chronic fatigue syndrome and fibromyalgia. In some aspects, the cancer can be ovarian cancer, breast cancer, pancreatic cancer, prostate cancer, lung cancer, hepatocellular carcinoma, acute myeloid leukemia, acute lymphoblastic leukemia, non-Hodgkin lymphomas, or Hodgkin lymphoma. In some aspects, the disease can be associated with a need to repress, block or inhibit FXII-mediated neutrophil activation.

Disclosed herein, are methods of treating a patient with type 1 diabetes, type 2 diabetes, venous thrombosis, arterial thrombosis, autoimmune diseases (e.g., systemic lupus erythematosus, Rheumatoid arthritis, psoriasis, acute liver toxicity (e.g., acetaminophen-induced acute liver injury), sickle cell disease and related acute hemolytic crisis, acute chest syndrome, and pulmonary artery thrombosis, and cancer. In some aspects, the methods of treating a patient with a disease caused by neutrophil activation or inflammatory diseases accompanied by neutrophil activation. In some aspects, the disease caused by neutrophil activation or inflammatory diseases accompanied by neutrophil activation can be venous and arterial thrombosis, deep vein thrombosis and vascular thrombo-embolism (DVT+VTE), lupus, psoriasis, atherosclerosis, endometriosis, trauma, sickle cell disease and associated acute hemolytic crisis, acute chest syndrome and pulmonary thrombosis, immunothrombosis, COVID-19 infection, thrombo-inflammation, chronic and diabetic wounds, post-operative wounds, trauma and related wounds, sepsis, acute respiratory distress syndrome, acute pancreatitis, acute pulmonary disorder, pulmonary disorder caused by the hemorrhagic shock, multiple organ failure, burn, multiple injury, idiopathic interstitial pulmonary fibrosis, cancer, cerebral trauma, spinal cord injury, neuropathic pain, cerebral infarction, cerebral vasospasm after the subarachnoid hemorrhage, epilepsy, status epilepticus, viral encephalitis, influenza-associated encephalopathy, inflammatory bowel disease, Kawasaki disease, multiple sclerosis, diabetic vascular complication, diabetic wounds, hepatitis, arteriosclerosis, asthma bronchial, chronic bronchitis, pulmonary emphysema, organ dysfunction after surgical operation, organ dysfunction after radiotherapy, nephritis, nephrotic syndrome, acute renal failure, hemodialysis, extracorporeal circulation, artificial breathing, acute/chronic rejection after organ transplantation, systemic lupus erythematosus (SLE), rheumatoid arthritis, disseminated intravascular coagulation (DIC), autoimmune disease group. Bechet's disease, myocarditis, endocarditis, ischemia reperfusion disorder, myocardial infarction, congestive heart failure, adipose tissue inflammation, neutrophilic dermatosis, Sweet's disease, Stevens-Johnson syndrome, Reye syndrome, cachexia, chronic fatigue syndrome and fibromyalgia. In some aspects, the cancer can be ovarian cancer, breast cancer, pancreatic cancer, prostate cancer, lung cancer, hepatocellular carcinoma, acute myeloid leukemia, acute lymphoblastic leukemia, non-Hodgkin lymphomas, or Hodgkin lymphoma. In some aspects, the disease can be associated with a need to repress, block or inhibit FXII-mediated neutrophil activation.

In some aspects, subject has ovarian cancer or is suspected of having ovarian cancer.

In some aspects, any of the methods disclosed herein can further comprise administering one or more of the following: antibiotics, topical dressings (e.g., hydrogels), chemotherapy agents (e.g., cisplatin, taxanes, anthracyclines, etoposide, vincristine), immune effector cells (e.g., CAR-T and NK cells), immunotherapy agents anti-angiogenesis agents (e.g., bevacizumab), anti-inflammatory agents (e.g., naproxen, celecoxib, ibuprofen), steroids (e.g., prednisone, dexamethasone, methylprednisolone, hydrocortisone), immunomodulating agents (e.g., monoclonal antibodies, TNF-α inhibitors, check point inhibitors), anticoagulation, NET degrading agents, DNase-1, anti-adhesion agents (e.g., crizanlizumab), statins, or Akt2 inhibitors to the subject.

Amounts effective for this use can depend on the severity of the disease and the weight and general state and health of the subject. Suitable regimes for initial administration and booster administrations are typified by an initial administration followed by repeated doses at one or more hourly, daily, weekly, or monthly intervals by a subsequent administration. For therapeutic uses, the peptides and compositions can include a pharmaceutically acceptable excipient. Such compositions can be formulated without undue experimentation for administration to a mammal, including humans, as appropriate for the particular application. Additionally, proper dosages of the compositions can be determined without undue experimentation using standard dose-response protocols. For example, a subject can receive any of the peptides or compositions disclosed herein one or more times per week (e.g., 2, 3, 4, 5, 6, or 7 or more times per week).

The total effective amount of any of peptides in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The therapeutically effective amount of any of the peptides disclosed herein present within the pharmaceutical compositions described herein and used in the methods as disclosed herein applied to mammals (e.g., humans) can be determined by one of ordinary skill in the art with consideration of individual differences in age, weight, and other general conditions (as mentioned herein).

### EXAMPLES

### Example 1: Determination of the UPAR Binding Site(s) on FXII and Design Inhibitory Peptides that Disrupt FXII-Mediated Neutrophil Activation

To identify the urokinase plasminogen activator receptor (uPAR) binding sites on Factor XII (FXII), 12 (FXII) deletion mutants were screened in protein-based assays including surface plasmon resonance and competitive binding assays. Each of these studies were performed with murine and human soluble uPAR. In both scenarios, considerable loss of FXII-uPAR binding was found in deletion mutants lacking the Fibronectin type II domain of FXII. Moreover, decreased binding between human FXII & uPAR in deletion mutants that lacked the proline-rich region of the heavy chain was found. Based on these results, the hydroxyl radical foot printing (HRF) technique was used to identify the binding interface of the FXII-uPAR protein complex.

Briefly, free uPAR and human FXII protein samples were prepared with the final protein concentration of 2.5 μM and 3 μM, respectively (1:1.2 uPAR: FXII molar ratio). Free uPAR and FXII and FXII-uPAR complex were exposed to hydroxyl radicals for intervals of 0, 12, 20, and 40 milliseconds using the X-ray beam line. Next, the irradiated samples were reduced and alkylated with 10 mM and 25 mM of DTT and iodoacetamide, respectively. Subsequently, the samples were subjected to a trypsin and AspN digestion (1:10 enzyme to protein ratio) followed by liquid chromatography coupled with high-resolution mass spectrometry (LC-MS). The MS data were analyzed manually resulting in dose-response plots for each peptide. The HRF process introduces stable side chain oxidative modifications resulting in specific mass shifts, which were identified from the tandem mass spectrometry data. The selected ion chromatograms (SIC) were extracted and integrated for the un-oxidized and the oxidized forms of peptide ion (with particular m z). These peak area values were used to characterize reaction kinetics in the form of dose-response (DR) plots, which measure the loss of intact peptide as a function of the hydroxyl radical exposure. The solvent-protected regions in the complex exhibit decreased oxidation reaction compared to the same regions in the free protein. Differences in the rate of oxidation (called rate constant, RC) indicate the potential locations of the binding interface. MSI data for free and complex proteins were used to determine RC values for each peptide within the protein. The overall fit results for peptides are shown in Table 4. Peptide location and the corresponding sequence are shown in columns 1 and 2, respectively. The third and fourth columns show the RC values for the free FXII protein and complex, respectively. The RC values were averaged from replicate experiments and the associated errors are shown next to each RC value. The fifth column shows the protection ratio, PR (=RC_{FXII}/RC_{Complex}). The sixth column shows the normalized protection ratio calculated as (mean+median)/2. Typically, a PR value close to 1 indicates that the solvent accessibility of the region remains unchanged while, a PR>1 indicates that the corresponding region exhibits protection from the solvent as a function of the complex formation. Based on these data, uPAR binding regions on FXII are shown in Table 4 (shown are FXII-binding sites for human uPAR, indicated in bold and underline).

TABLE 4
Modification rates for specific amino acids from free FXII protein against
FXII in complex with human uPAR.

| | | | Modification | Modification | | |
|---|---|---|---|---|---|---|
| | | | rate, | rate, | | Normalized |
| | | | K_{FXII}, S⁻¹ | K_{COM}, S⁻¹ | Protection | ratio |
| Position | Sequence, FXII | Site | FXII free | FXII complex | Ratio, PR | (PR/1.25) |
| 1-8 | IPPWEAPK | W4 | 3.08 ± 0.25 | 3.08 ± 0.13 | 2.01 | 1.61 |
| | (SEQ ID NO: 1) | | | | | |
| 46-60 | GRPGPQPWCATTPNF | P50 | 0.25 ± 0.019 | 0.16 ± 0.013 | 1.56 | 1.25 |
| | (SEQ ID NO: 7) | W53 | 1.12 ± 0.12 | 0.75 ± 0.04 | 1.49 | 1.19 |
| | | P58 | 0.14 ± 0.016 | 0.14 ± 0.0082 | 1.0 | 0.8 |
| | | F60 | 0.026 ± 0.0015 | 0.025 ± 0.0020 | 1.04 | 0.83 |
| 272-291 | DLAQCQTPTQAAPPT | Q281 | 0.97 ± 0.047 | 0.53 ± 0.019 | 1.83 | 1.46 |
| | PVSPR | P285 | 1.3 ± 0.061 | 0.87 ± 0.030 | 1.49 | 1.19 |
| | (SEQ ID NO: 2) | P287 | 0.23 ± 0.021 | 0.11 ± 0.009 | 2.09 | 1.67 |
| | | V288 | 0.71 ± 0.04 | 0.46 ± 0.019 | 1.54 | 1.23 |
| 292-305 | LHVPLMPAQPAPPK | H293 | 2.55 ± 0.30 | 1.82 ± 0.15 | 1.40 | 1.12 |
| | (SEQ ID NO: 3) | V294 | 0.85 ± 0.093 | 0.79 ± 0.078 | 1.08 | 0.86 |
| | | L296 | 0.62 ± 0.054 | 0.29 ± 0.057 | 2.14 | 1.71 |
| | | K305 | 1.06 ± 0.063 | 0.58 ± 0.042 | 1.83 | 1.46 |

Site-directed mutagenesis (Ala substitutions) was performed in those FXII amino acids that exhibited the highest modification rates between free FXII and FXII-uPAR conformations (FIG. 1C, right panel). Each variant was characterized in several assays including microscale thermophoresis. The results demonstrate that uPAR directly interacts with a continuous stretch of amino acids in the kringle and proline-rich regions of FXII. Mutating 4 residues within the KR (variant 4A representing amino acids 281, 285, 287, 288: SEQ ID NO: 2) results in sequential loss of the FXII-uPAR interaction (FIG. 1D). Importantly, single substitution of W residue at position 4 of the intact zymogen FXII (W4A variant: SEQ ID NO: 1), resulted in significantly reduced FXII-uPAR binding (FIG. 1D). Therefore, the results show that side chain flexibility of the FN II region maintains quiescence of uPAR binding sites that span the KR and PR regions. These findings explain, at least in part, why the FXII-uPAR interaction is not a constitutive process in vivo.

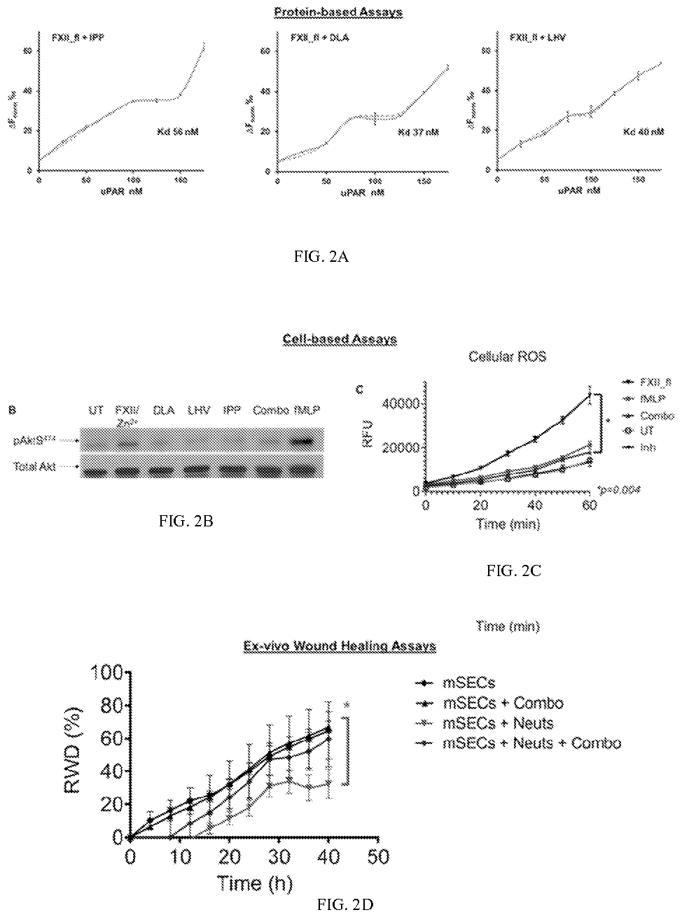

The results provide insight on the structure of the non-catalytic region of FXII. These advances have also provided the structural details required to develop peptide antagonists. To this end, 3 peptides containing FXII amino acid residues implicated in uPAR binding were synthesized and termed “IPP” (corresponding to SEQ ID NO: 1), “DLA” (corresponding to SEQ ID NO: 2), and “LHV” (corresponding to SEQ ID NO: 3). The kinetics of peptide interference with FXII-uPAR binding was screened which yielded nanomolar affinity for each (FIG. 2A).

Finally, the effects of FXII-derived peptides were characterized on relevant neutrophil functions (pAkt2 formation and ROS generation, both implicated in NET formation and impaired wound healing: FIGS. 2B-C), as well as ex vivo, in a wound simulating environment. Live video microscopy of wounded keratinocytes was employed, in the absence or presence of neutrophils and combination peptides, neutrophil signaling and ROS assays. The results demonstrate that neutrophils significantly impair keratinocyte migration: however, pre-incubating neutrophils with combination of IPP-DLA-LHV (10 μM) peptides, significantly accelerated wound closure (FIG. 2D).

### Example 2: Targeting FXII-Mediated Signaling Reverses Pro-Invasive Tumor Traits

Based on these results described in Example 1, it was tested whether targeting FXII-mediated signaling can be beneficial in states where persistent neutrophil responses have been linked to disease, for example, diabetic wounds. When non-diabetic and diabetic neutrophils from WT and FXII deficient mice were examined in the same wound assay, it was found that in contrast to DM WT neutrophils which significantly impair keratinocyte migration, FXII deficient neutrophils were significantly less inhibitory.

Tumor behavior in vivo represents a chronic, non-healing wound. Tumors that grow progressively in the host have developed the capacity to continuously initiate the wound healing response of the host as a means to acquire the stroma they need to grow and expand. However, in contrast to wounds, this process is not self-limited. In this context, it was assessed whether FXII influences tumor behavior. Epithelial ovarian cancer (EOC) was used in these studies because tumor spread is not hematogenous but rather, proceeds intraperitoneally by tissue proteolysis (where uPAR is involved) and, clinical studies reporting that the degree of neutrophil activation in peripheral blood correlated with adverse prognosis in women with EOC.

The focus of the study was whether FXII and uPAR contribute to neutrophil-induced epithelial-to-mesenchymal transition (EMT). FXII KO neutrophils from tumor-bearing mice were used, and they were co-cultured ex vivo with ID8 tumor cells to determine the effects on EMT. Next, a combination of inhibitory peptides described herein that were developed that specifically abrogate the FXII-uPAR interaction (combination of IPP, DLA and LHV peptides) were used. Co-culture of neutrophils from WT tumor-bearing cells with ID8 tumor cells led to significant increase in vimentin expression compared to tumor cells alone. Strikingly, use of inhibitory peptides reversed the pro-invasive phenotype of both tumor cells and WT neutrophils. These studies also provided evidence that tumor FXII-uPAR are functional and contribute to tumor cell pro-invasive traits. In contrast, co-culture of tumor cells with FXII KO neutrophils, did not promote an increase in vimentin expression and use of a combination of FXII inhibitory peptides increased E-cadherin expression. At the mRNA level, it was confirmed that loss of FXII expression does not accentuate the expression of mesenchymal vimentin and N-cadherin. In sum, these studies show that the FXII-uPAR axis is operative in both tumor cells and neutrophils where it contributes to a mesenchymal and migratory phenotype and abrogating FXII-mediated signaling effectively reverses these pro-invasive traits.

### Example 3: Targeted Inhibition of the FXII-uPAR-pAkt2 Axis is Therapeutically Effective in Treating DVT while Minimizing Systemic Side-Effects and Bleeding Risk

Historically, the pathogenesis of deep vein thrombosis (DVT) has been described by Virchow's triad, which proposes that three major factors contribute to venous thromboembolism (VTE): (1) reduction in blood flow (stasis), (2) injury to the vascular endothelium, and (3) the presence of a hypercoagulable state. This paradigm shifted with the observation in recent years that neutrophils significantly contribute to thrombosis, termed thromboinflammation.

The cooperation of platelets with neutrophils was identified using a murine model of DVT in which flow restriction induces thrombosis in the inferior vena cava (IVC). In this model, platelets and neutrophils are promptly recruited to the vessel wall within hours of reduced blood flow and engage in heterotypic cell-cell interactions.

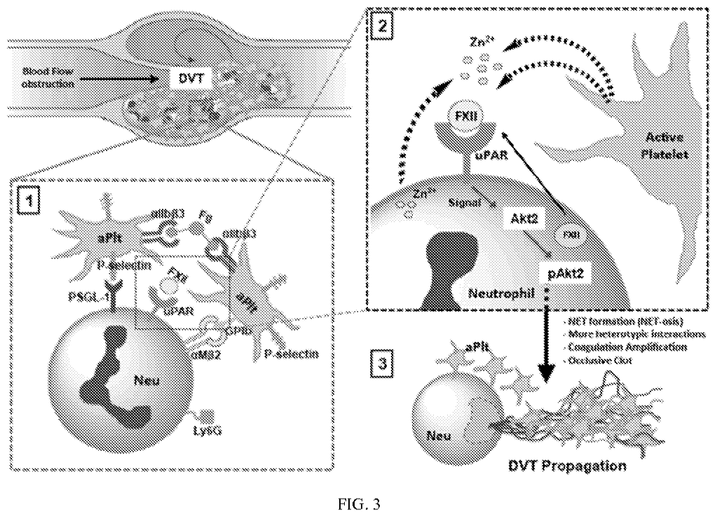

These interactions facilitate DVT growth and propagation by: 1) supporting additional neutrophil recruitment; and 2) stimulating neutrophils to release NETs which act as prothrombotic scaffolds leading to coagulation factor assembly and fibrin formation (FIG. 3). Neutrophil Akt2 phosphorylation (pAkt2) is important for such NET formation and upregulates the membrane translocation of neutrophil αMβ2 integrin, to further amplify and stabilize neutrophil-platelet interactions. In this framework, it was found that FXII engages uPAR on the neutrophil surface to promote Akt2 phosphorylation in a Zinc (Zn2+)-dependent manner. In fact, genetic deletion or pharmacologic inhibition of FXII resulted in decreased neutrophil influx at DVT sites, decreased NETosis, and smaller IVC thrombi. These FXII functions on neutrophils are independent and different from its enzymatic activity in the intrinsic pathway of coagulation.

The recognition that excess neutrophil activation contributes to pathological thrombosis has led to the development of agents that modulate neutrophil functions as a treatment for DVT. These strategies include neutrophil depletion, induction of neutrophil apoptosis, or dissolution of NETs. However, pre-clinical and clinical studies revealed significant challenges with these therapeutic approaches that relate to 1) inhibiting important neutrophil functions (i.e., innate immunity), 2) the limited half-life of agents, and 3) off-target effects associated with systemic delivery. Therefore, targeted delivery of therapeutic agents towards DVT-associated neutrophils provide effective drug half-life and minimal systemic side-effects.

As described herein, FXII-uPAR upregulates neutrophil functions. Specifically, it has shown that following neutrophil activation, autocrine FXII signals through uPAR leading to phosphorylation of Akt2 and to neutrophil adhesion, chemotaxis, and NET formation (FIG. 3). Inhibiting FXII signaling in neutrophils resulted in smaller venous thrombi. Based on these mechanistic findings, that the results described herein show that targeted inhibition of the FXII-uPAR-pAkt2 axis can be therapeutically effective in treating DVT while minimizing systemic side-effects and bleeding risk.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A peptide comprising an amino acid sequence of at least 60% identity to the amino acid sequence of IPPWEAPK (SEQ ID NO: 1) or a retro-inverso amino acid sequence of SEQ ID NO: 1, wherein the peptide binds urokinase-type plasminogen activator receptor (uPAR).

2. The peptide of claim 1, wherein the peptide comprises an amino acid sequence of at least 70% identity to the amino acid sequence of IPPWEAPK (SEQ ID NO: 1) or a retro-inverso amino acid sequence of SEQ ID NO: 1.

3. The peptide of claim 1 or 2, wherein peptide comprises an amino acid sequence comprising a W at position 4 of SEQ ID NO: 1.

4. The peptide of claim 1, wherein the peptide is IPPWEAPK (SEQ ID NO: 1).

5. A peptide comprising an amino acid sequence of at least 60% identity to the amino acid sequence of DLAQCQTPTQAAPPTPVSPR (SEQ ID NO: 2) or a retro-inverso amino acid sequence of SEQ ID NO: 2, wherein the peptide binds urokinase-type plasminogen activator receptor (uPAR).

6. The peptide of claim 5, comprising an amino acid sequence of at least 70% identity to the amino acid sequence of DLAQCQTPTQAAPPTPVSPR (SEQ ID NO: 2) or a retro-inverso amino acid sequence of SEQ ID NO: 2.

7. The peptide of claim 6 or 7, wherein the amino acid sequence comprises a Q at position 10, a P at position 14, a P at position 16, and a V at position 17 of SEQ ID NO: 2.

8. The peptide of claim 5, wherein the peptide is DLAQCQTPTQAAPPTPVSPR (SEQ ID NO: 2).

9. A peptide comprising an amino acid sequence of at least 60% identity to the amino acid sequence of LHVPLMPAQPAPPK (SEQ ID NO: 3) or a retro-inverso amino acid sequence of SEQ ID NO: 3, wherein the peptide binds urokinase-type plasminogen activator receptor (uPAR).

10. The peptide of claim 9, comprising an amino acid sequence of at least 70% identity to the amino acid sequence of LHVPLMPAQPAPPK (SEQ ID NO: 3) or a retro-inverso amino acid sequence of SEQ ID NO: 3.

11. The peptide of claim 9 or 10, wherein the amino acid sequence comprises a H at position 2, a V at position 3, a M at position 6, or a K at position 14 of SEQ ID NO: 3.

12. The peptide of claim 9, wherein the peptide is LHVPLMPAQPAPPK (SEQ ID NO: 3).

13. The peptide of any of the preceding claims, wherein the peptide is linear or cyclized.

14. The peptide of claim 13, wherein the peptide is cyclized via a disulfide bridge between terminal cysteine residues.

15. The peptide of claim 13, wherein the peptide is cyclized

16. The peptides of any of the preceding claims, wherein the peptide comprises one or more polyethylene glycol moieties (PEG).

17. A method of reducing neutrophil activation in a subject, the method comprising: a) administering to the subject a therapeutically effective amount of any of the peptides of any of claims 1-16; and b) a pharmaceutically acceptable carrier.

18. A method of reducing reaction oxygen species production in a subject, the method comprising: a) administering to the subject a therapeutically effective amount of any of the peptides of any of claims 1-16; and b) a pharmaceutically acceptable carrier.

19. A method of reducing production of neutrophil extracellular traps in a subject, the method comprising: a) administering to the subject a therapeutically effective amount of any of the peptides of any of claims 1-16; and b) a pharmaceutically acceptable carrier.

20. A method of improving wound closure in a subject, the method comprising: a) administering to the subject a therapeutically effective amount of any of the peptides of any of claims 1-16; and b) a pharmaceutically acceptable carrier.

21. A method of reducing vimentin levels in a subject, the method comprising: a) administering to the subject a therapeutically effective amount of any of the peptides of any of claims 1-16; and b) a pharmaceutically acceptable carrier.

22. A method of reducing epithelial-to-mesenchymal transition in a subject, the method comprising: a) administering to the subject a therapeutically effective amount of any of the peptides of any of claims 1-16; and b) a pharmaceutically acceptable carrier.

23. The method of any of claims 17 to 22, wherein the subject is identified as being in need of treatment before the administration step.

24. The method of any of claims 17 to 22, wherein the subject is a human.

25. The method of any of claims 17 to 22, wherein the composition is formulated for intravenous, subcutaneous, intradermal, intraperitoneal, intraocular, or intravitreal administration.

26. The method of claim 20, wherein the subject has type 1 or 2 diabetes, acute liver toxicity, venous thrombosis, arterial thrombosis, deep vein thrombosis and vascular thrombo-embolism (DVT+VTE), lupus, psoriasis, atherosclerosis, endometriosis, trauma, sickle cell disease and associated acute hemolytic crisis, acute chest syndrome and pulmonary thrombosis, immunothrombosis, COVID-19 infection, thrombo-inflammation, chronic and diabetic wounds, post-operative wounds, trauma and related wounds, sepsis, acute respiratory distress syndrome, acute pancreatitis, acute pulmonary disorder, pulmonary disorder caused by the hemorrhagic shock, multiple organ failure, burn, multiple injury, idiopathic interstitial pulmonary fibrosis, cerebral trauma, spinal cord injury, neuropathic pain, cerebral infarction, cerebral vasospasm after the subarachnoid hemorrhage, epilepsy, status epilepticus, viral encephalitis, influenza-associated encephalopathy, inflammatory bowel disease, Kawasaki disease, multiple sclerosis, diabetic vascular complication, diabetic wounds, hepatitis, arteriosclerosis, asthma bronchial, chronic bronchitis, pulmonary emphysema, organ dysfunction after surgical operation, organ dysfunction after radiotherapy, nephritis, nephrotic syndrome, acute renal failure, hemodialysis, extracorporeal circulation, artificial breathing, acute/chronic rejection after organ transplantation, systemic lupus erythematosus (SLE), rheumatoid arthritis, disseminated intravascular coagulation (DIC), autoimmune disease group, Bechet's disease, myocarditis, endocarditis, ischemia reperfusion disorder, myocardial infarction, congestive heart failure, adipose tissue inflammation, neutrophilic dermatosis, Sweet's disease, Stevens-Johnson syndrome, Reye syndrome, cachexia, chronic fatigue syndrome, fibromyalgia, ovarian cancer, breast cancer, pancreatic cancer, prostate cancer, lung cancer, hepatocellular carcinoma, acute myeloid leukemia, acute lymphoblastic leukemia, non-Hodgkin lymphomas, or Hodgkin lymphoma.

27. The method of claim 21, wherein the subject has ovarian cancer or is suspected of having ovarian cancer.

28. The method of any of claims 17 to 22, further comprising administering antibiotics, topical dressings, chemotherapy agents, immune effector cells, immunotherapy agents anti-angiogenesis agents, anti-inflammatory agents, steroids, immunomodulating agents, anticoagulation, NET degrading agents, DNase-1, anti-adhesion agents, statins, Akt2 inhibitors to the subject.


SEQUENCE LISTING (11 sequences)

SEQ ID NO: 1 - protein, 8 aa, synthetic construct
  REGION 1..8: note = Synthetic construct
IPPWEAPK

SEQ ID NO: 2 - protein, 20 aa, synthetic construct
  REGION 1..20: note = Synthetic construct
DLAQCQTPTQAAPPTPVSPR

SEQ ID NO: 3 - protein, 14 aa, synthetic construct
  REGION 1..14: note = Synthetic construct
LHVPLMPAQPAPPK

SEQ ID NO: 4 - skipped

SEQ ID NO: 5 - protein, 20 aa, synthetic construct
  REGION 1..20: note = Synthetic construct
  VARIANT 10: note = MISC_FEATURE - A or R or N or D or C or Q or E or G or H or I or L or K or M or F or P or O or S or U or T or W or Y or V
  VARIANT 14: note = MISC_FEATURE - A or R or N or D or C or Q or E or G or H or I or L or K or M or F or P or O or S or U or T or W or Y or V
  VARIANT 16: note = MISC_FEATURE - A or R or N or D or C or Q or E or G or H or I or L or K or M or F or P or O or S or U or T or W or Y or V
  VARIANT 17: note = MISC_FEATURE - A or R or N or D or C or Q or E or G or H or I or L or K or M or F or P or O or S or U or T or W or Y or V
DLAQCQTPTXAAPXTXXSPR

SEQ ID NO: 6 - protein, 14 aa, synthetic construct
  REGION 1..14: note = Synthetic construct
  VARIANT 2: note = MISC_FEATURE - A or R or N or D or C or Q or E or G or H or I or L or K or M orF or P or O or S or U or T or W or Y or V
  VARIANT 3: note = MISC_FEATURE - A or R or N or D or C or Q or E or G or H or I or L or K or M orF or P or O or S or U or T or W or Y or V
  VARIANT 5: note = MISC_FEATURE - A or R or N or D or C or Q or E or G or H or I or L or K or M orF or P or O or S or U or T or W or Y or V
  VARIANT 6: note = MISC_FEATURE - A or R or N or D or C or Q or E or G or H or I or L or K or M orF or P or O or S or U or T or W or Y or V
  VARIANT 14: note = MISC_FEATURE - A or R or N or D or C or Q or E or G or H or I or L or K or M orF or P or O or S or U or T or W or Y or V
LXXPXXPAQPAPPX

SEQ ID NO: 7 - protein, 615 aa, synthetic construct
  REGION 1..615: note = Synthetic construct
MRALLLLGFLLVSLESTLSIPPWEAPKEHKYKAEEHTVVLTVTGEPCHFPFQYHRQLYHKCTHKGRPGPQPWCATTPNFDQDQRWGYCLEPKKVKDHCSKHSPCQKGGTCVNMPSGPHCLCPQHLTGNHCQKEKCFEPQLLRFFHKNEIWYRTEQAAVARCQCKGPDAHCQRLASQACRTNPCLHGGRCLEVEGHRLCHCPVGYTGAFCDVDTKASCYDGRGLSYRGLARTTLSGAPCQPWASEATYRNVTAEQARNWGLGGHAFCRNPDNDIRPWCFVLNRDRLSWEYCDLAQCQTPTQAAPPTPVSPRLHVPLMPAQPAPPKPQPTTRTPPQSQTPGALPAKREQPPSLTRNGPLSCGQRLRKSLSSMTRVVGGLVALRGAHPYIAALYWGHSFCAGSLIAPCWVLTAAHCLQDRPAPEDLTVVLGQERRNHSCEPCQTLAVRSYRLHEAFSPVSYQHDLALLRLQEDADGSCALLSPYVQPVCLPSGAARPSETTLCQVAGWGHQFEGAEEYASFLQEAQVPFLSLERCSAPDVHGSSILPGMLCAGFLEGGTDACQGDSGGPLVCEDQAAERRLTLQGIISWGSGCGDRNKPGVYTDVAYYLAWIREHTVS

SEQ ID NO: 8 - protein, 335 aa, synthetic construct
  REGION 1..335: note = Synthetic construct
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 9 - protein, 596 aa, synthetic construct
  REGION 1..596: note = Synthetic construct
IPPWEAPKEHKYKAEEHTVVLTVTGEPCHFPFQYHRQLYHKCTHKGRPGPQPWCATTPNFDQDQRWGYCLEPKKVKDHCSKHSPCQKGGTCVNMPSGPHCLCPQHLTGNHCQKEKCFEPQLLRFFHKNEIWYRTEQAAVARCQCKGPDAHCQRLASQACRTNPCLHGGRCLEVEGHRLCHCPVGYTGAFCDVDTKASCYDGRGLSYRGLARTTLSGAPCQPWASEATYRNVTAEQARNWGLGGHAFCRNPDNDIRPWCFVLNRDRLSWEYCDLAQCQTPTQAAPPTPVSPRLHVPLMPAQPAPPKPQPTTRTPPQSQTPGALPAKREQPPSLTRNGPLSCGQRLRKSLSSMTRVVGGLVALRGAHPYIAALYWGHSFCAGSLIAPCWVLTAAHCLQDRPAPEDLTVVLGQERRNHSCEPCQTLAVRSYRLHEAFSPVSYQHDLALLRLQEDADGSCALLSPYVQPVCLPSGAARPSETTLCQVAGWGHQFEGAEEYASFLQEAQVPFLSLERCSAPDVHGSSILPGMLCAGFLEGGTDACQGDSGGPLVCEDQAAERRLTLQGIISWGSGCGDRNKPGVYTDVAYYLAWIREHTVS

SEQ ID NO: 10 - protein, 313 aa, synthetic construct
  REGION 1..313: note = Synthetic construct
LRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 11 - protein, 20 aa, synthetic construct
  REGION 1..20: note = Synthetic construct
LHVPLMPAQPAPPKPQPTTR